(19) United States
(12) Patent Application Publication
(10) Pub. No.: US 2005/0176733 A1
(43) Pub. Date: Aug. 11, 2005

(54) Chk-1 inhibitors

(75) Inventors: Robert G. Boyle, Cambridge (GB); Hassan Julien Imogai, Geneva (CH); Michael Cherry, Cambridge (GB); Nawaz Mohammed Khan, Cambridge (GB)

Correspondence Address: MILLENNIUM PHARMACEUTICALS, INC., 40 Landsdowne Street, CAMBRIDGE, MA, 02139, US

(73) Assignee: Millennium Pharmaceuticals, Inc., Cambridge, MA (US)

(21) Appl. No.: 11/031,544

(22) Filed: Jan. 7, 2005

Related U.S. Application Data
(60) Provisional application No. 60/537,523, filed on Jan. 20, 2004.

(51) Int. Cl. A61K 31/497; A61K 31/444; C07D 43/02; C07D 41/02

(52) U.S. Cl. 514/255.05; 514/332; 544/405; 546/265

(57) ABSTRACT

This invention provides compounds and methods for treating cancer. More particularly, the invention provides compounds that inhibit Chk-1. These compounds potentiate the action of DNA-damaging agents such as chemotherapy and radiation therapy.

This application claims priority to U.S. provisional application No. 60/537,523, filed on Jan. 20, 2004, the contents of which are incorporated herein by reference.

### BACKGROUND OF THE INVENTION

Cell cycle checkpoints are regulatory pathways that control the order and timing of cell cycle transitions. They ensure that critical events such as DNA replication and chromosome segregation are completed in high fidelity. The regulation of these cell cycle checkpoints is a critical determinant of the manner in which tumor cells respond to many chemotherapies and radiation. Many effective cancer therapies work by causing DNA damage; however, resistance to these agents remains a significant limitation in the treatment of cancer. Of the several mechanisms of drug resistance, an important one is attributed to the prevention of cell cycle progression through the control of critical activation of a checkpoint pathway. This arrests the cell cycle to provide time for repair, and induces the transcription of genes to facilitate repair, thereby avoiding immediate cell death. By abrogating checkpoint arrests at, for example, the S and G2 checkpoint, it may be possible to synergistically augment tumor cell death induced by DNA damage and circumvent resistance. (Shyjan et al., U.S. patent application Ser. No. 09/340,264 (1999)). Human Chk-1 plays a role in regulating cell cycle arrest by phosphorylating the phosphatase cdc25 on Serine 216, which may be involved in preventing activation of cdc2/cyclin B and initiating mitosis. (Sanchez et al., Science, 277:1497 (1997)). Therefore, inhibition of Chk-1 should enhance the effects of DNA damaging agents by initiating mitosis before DNA repair is complete and thereby causing tumor cell death.

### SUMMARY OF THE INVENTION

The present invention provides compounds that are effective inhibitors of Chk-1. These compounds are useful for the treatment of cancer, particularly when used in combination with DNA-damaging agents.

The Chk-1 inhibitors of the present invention have the formula (I): 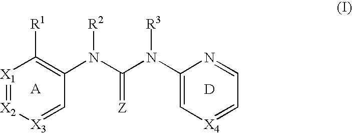
or a pharmaceutically acceptable salt thereof, wherein:
X₁-X₃ are independently CH or N, provided that X₁-X₃ are not all N;
X₄ is CH or N;
Z is O, S, N—R, or N—CN;
Ring A is optionally substituted at any substitutable carbon by R⁴;
Ring D is optionally substituted by C₁, aliphatic or haloaliphatic, —OR⁷, —SR⁷, —C(O)R, —CO₂R⁷, —SO₂R, —CN, —C(O)N(R⁷)₂, —N(R⁷)C(O)R, or —N(R⁷)₂, and is optionally fused to an optionally substituted phenyl or optionally substituted cyclohexyl ring;
R¹ is -T-W or -V-T-W;
T is a C₁₋₆ straight or branched alkylidene chain that is optionally substituted by F, —OR⁶, —N(R⁶)₂, or —CO₂R⁶, and is optionally interrupted by —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—, wherein the alkylidene chain or a portion thereof is optionally part of a 3-6 membered ring system;
V is —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—;
W is —C(O)N(R⁹)₂, —N(R⁶)—C(O)—R¹⁰, —N(R⁶)—C(O)—N(R⁹)₂, —N(R⁶)—C(O)—OR¹¹, —O—C(O)—N(R⁹)₂, —NH—C(═NH)—R¹⁰, or —NH—(═NH)—NH—R⁹;
each of R² and R³ independently is hydrogen or C₁₋₆ alkyl optionally substituted with —N(R⁸)₂, —C(O)R, —CO₂R, or SO₂R; or R² and R³, taken together with the intervening atoms, form an optionally substituted 5-6 membered ring;
each R⁴ independently is halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, or an optionally substituted C₁₋₆ aliphatic, aryl, aralkyl, heterocyclyl, heterocyclylalkyl, heteroaryl, or heteroaralkyl group; or two adjacent R⁴, taken together, form an optionally substituted phenyl, pyridyl or heterocyclyl ring fused to Ring A;
each R⁵ independently is hydrogen, C₁₋₆ aliphatic, —CO₂R, —SO₂R, or —C(O)R; or —N(R⁵)₂ is an optionally substituted nitrogen-containing heterocyclyl;
each R⁶ independently is hydrogen or an optionally substituted C₁₋₃ aliphatic;
each R⁷ independently is hydrogen or an optionally substituted C₁₋₃ aliphatic; or —N(R⁷)₂ is an optionally substituted nitrogen-containing heterocyclyl;
each R⁸ independently is a C₁₋₃ alkyl; or —N(R⁸)₂ is an optionally substituted nitrogen-containing heterocyclyl;
each R⁹ independently is hydrogen, an optionally substituted C₁₋₆ aliphatic, an optionally substituted heterocyclyl, or an optionally substituted heteroaryl; or —N(R⁹)₂ is an optionally substituted nitrogen-containing heterocyclyl;
each R¹⁰ independently is an optionally substituted aryl, heteroaryl, heterocyclyl, or C₁₋₆ aliphatic group;
R¹¹ is an optionally substituted aryl, heteroaryl, heterocyclyl, or C₁₋₆ aliphatic, group, provided that R¹¹ is other than tert-butyl or arylmethyl; and
each R independently is hydrogen or an optionally substituted C₁₋₆ aliphatic, aryl, aralkyl, heteroaryl, or heteroaralkyl group.

The invention also provides a pharmaceutical composition comprising one or more compounds of formula (I) and a pharmaceutically acceptable carrier. In some embodiments, the pharmaceutical composition additionally comprises a radiotherapeutic or chemotherapeutic agent.

The invention further provides a method for inhibiting Chk-1, comprising contacting a Chk-1 enzyme with one or more compounds of formula (I) or a pharmaceutically acceptable salt thereof.

The invention further provides a method for inhibiting Chk-1 in a subject in need of such inhibition comprising administering to the subject an effective amount of a compound of formula (I) or a pharmaceutically acceptable salt thereof.

The invention further provides a method of treating cancer in a subject, comprising administering to a subject in need of such treatment an effective amount of a compound of formula (I) or a pharmaceutically acceptable salt thereof. In some embodiments the method further comprises administering a DNA damaging agent. Also contemplated within the scope of the invention is the use of a compound of formula (I) for the manufacture of a medicament of inhibiting Chk-1 in a subject in need of such inhibition or for treating a subject with cancer.

The invention further provides a kit comprising (i) a Chk-1 inhibitor of formula (I) or a pharmaceutically acceptable salt thereof; and (ii) a package insert comprising instructions for administering to a subject the Chk-1 inhibitor and a DNA damaging agent.

The invention further provides a kit comprising (i) a DNA damaging agent; and (ii) a package insert comprising instructions for administering to a subject the DNA damaging agent and a Chk-1 inhibitor of formula (I) or a pharmaceutically acceptable salt thereof.

### DESCRIPTION OF THE INVENTION

This invention provides compounds and methods for treating cancer. More particularly, the invention provides compounds that inhibit Chk-1. These compounds potentiate the action of DNA-damaging agents such as chemotherapy and radiation therapy.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. The issued patents, applications, and references that are cited herein are hereby incorporated by reference to the same extent as if each was specifically and individually indicated to be incorporated by reference. In the case of inconsistencies, the present disclosure, including definitions, will control. In addition, the materials, methods, and examples are illustrative only and are not intended to be limiting.

The Chk-1 inhibitors of the present invention have the formula (I): 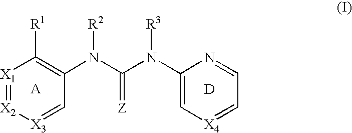
or a pharmaceutically acceptable salt thereof, wherein:
X₁-X₃ are independently CH or N, provided that X₁-X₃ are not all N;
X₄ is CH or N;
Z is O, S, N—R, or N—CN;
Ring A is optionally substituted at any substitutable carbon by R⁴;
Ring D is optionally substituted by C₁₋₄ aliphatic or haloaliphatic, —OR⁷, —SR⁷, —C(O)R, —C₂R⁷, —SO₂R, —CN, —C(O)N(R⁷)₂, —N(R⁷)C(O)R, or —N(R⁷)₂, and is optionally fused to an optionally substituted phenyl or optionally substituted cyclohexyl ring;
R¹ is -T-W or -V-T-W;
T is a C₁₋₆ straight or branched alkylidene chain that is optionally substituted by F, —OR⁶, —N(R⁶)₂, or —CO₂R⁶, and is optionally interrupted by —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—, wherein the alkylidene chain or a portion thereof is optionally part of a 3-6 membered ring system;
V is —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—;
W is —C(O)N(R⁹)₂, —N(R⁶)—C(O)—R¹⁰, —N(R⁶)—C(O)—N(R⁹)₂, —N(R⁶)—C(O)—OR¹¹, —O—C(O)—N(R⁹)₂, —NH—C(═NH)—R¹⁰, or —NH—(═NH)—NH—R⁹;
each of R² and R³ independently is hydrogen or C₁₋₆ alkyl optionally substituted with —N(R⁸)₂, —C(O)R, —CO₂R, or SO₂R; or R² and R³, taken together with the intervening atoms, form an optionally substituted 5-6 membered ring;
each R⁴ independently is halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, or an optionally substituted C₁₋₈, aliphatic, aryl, aralkyl, heterocyclyl, heterocyclylalkyl, heteroaryl, or heteroaralkyl group; or two adjacent R⁴, taken together, form an optionally substituted phenyl, pyridyl or heterocyclyl ring fused to Ring A;
each R⁵ independently is hydrogen, C₁₋₆ aliphatic, —CO₂R, —SO₂R, or —C(O)R; or —N(R⁵)₂ is an optionally substituted nitrogen-containing heterocyclyl;
each R⁶ independently is hydrogen or an optionally substituted C₁₋₃ aliphatic;
each R⁷ independently is hydrogen or an optionally substituted C₁₋₃ aliphatic; or —N(R⁷)₂ is an optionally substituted nitrogen-containing heterocyclyl;
each R⁸ independently is a C₁₋₃ alkyl; or —N(R⁸)₂ is an optionally substituted nitrogen-containing heterocyclyl;
each R⁹ independently is hydrogen, an optionally substituted C₁₋₆ aliphatic, an optionally substituted heterocyclyl, or an optionally substituted heteroaryl; or —N(R⁹)₂ is an optionally substituted nitrogen-containing heterocyclyl;
each R¹⁰ independently is an optionally substituted aryl, heteroaryl, heterocyclyl, or C₁₋₆ aliphatic group;
R¹¹ is an optionally substituted aryl, heteroaryl, heterocyclyl, or C₁₋₆ aliphatic, group, provided that R¹¹ is other than tert-butyl or arylmethyl; and
each R independently is hydrogen or an optionally substituted C₁₋₆ aliphatic, aryl, aralkyl, heteroaryl, or heteroaralkyl group.

In some embodiments, the invention relates to a compound of formula (I), wherein Ring D has the formula: 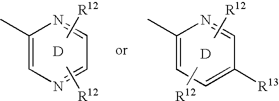
where:
each R¹² independently is selected from hydrogen, C₁₋₄ aliphatic or haloaliphatic, —OR⁷, —SR⁷, —C(O)R, —CO₂R⁷, —SO₂R, —CN, —C(O)N(R⁷)₂, —N(R⁷)C(O)R, or —N—(R⁷)₂; or two adjacent R¹² together form an optionally substituted fused phenyl or cyclohexyl ring; and
R¹³ is CO₂R⁷, —CN, or —C(O)N(R⁷)₂; and
R⁷ is as defined above.

In certain preferred embodiments, Ring D is selected from the group consisting of 2-pyrazinyl, 5-methyl-pyrazin-2-yl, 5-cyano-pyrazin-2-yl, 5-cyano-pyridin-2-yl, 5-carbamoyl-pyridin-2-yl, and 5-carbamoyl-pyrazin-2-yl.

In some embodiments, each of X₁—X₃ is CH, wherein one H in X₁—X₃ optionally is replaced with a substituent R⁴, as defined above. In some embodiments, Ring A has the formula:

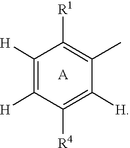

In certain preferred embodiments, Ring A has the formula shown above, wherein R⁴ is selected from the group consisting of —F, —Cl, —Br, —I, —COOR^{a}, —NHCOR^{a}, —CF₃, —CH₃, —CH₂CH₃, —CH(CH₃)₂, —N(R^{a})₂, —CH₂N(R^{a})₂, —CH₂CH₂N(R^{a})₂, piperidinyl, morpholinyl and pyrrolidinyl; and R^{a} is —CH₃, —CH₂CH, —CH₂CH₂NH₂, —CH₂CH₂NH(CH₃), —CH₂CH₂N(CH₃)₂, —CH₂CH₂(N-morpholinyl), —CH₂CH₂(N-piperidinyl) or —CH₂CH₂(N-pyrrolidinyl).

In some embodiments, the invention relates to a compound of formula (I), wherein V is —O— and T is a straight or branched C₂₋₅ alkylidene chain. In some such embodiments, T is a straight or branched C₂ or C₃ alkylidene chain.

In some embodiments, the invention relates to a compound of formula (I), having one or more features selected from the group consisting of:
(a) R² and R³ are each hydrogen;
(b) Z is oxygen;
(c) each of X₁—X₃ is CH;
(d) V is —O—, and T is a straight or branched C₂₋₅ alkylidene chain; and
(e) W is —C(O)N(R⁹)₂, —NH—C(O)—R¹⁰, —NH—C(O)—N(R⁹)₂, —NH—C(O)—OR¹¹, —O—C(O)—N(R⁹)₂, —NH—C(═NH)—R¹⁰, or —NH—(═NH)—NH—R⁹.

In various embodiments, at least one, two, three, or four of the above features are present. In a particularly preferred embodiment, the Chk-1 inhibitor of the invention has all five of the above features (a)-(e).

In some embodiments, R⁶ is hydrogen or a C₁₋₃ aliphatic that is optionally substituted with one to three, preferably one or two, substituents selected from the group consisting of -fluoro, —OR, —CN, —CO₂R⁷, —N(R⁷)₂, and optionally substituted C₆₋₁₀ aryl. In some embodiments, R⁶ is hydrogen or a C₁₋₃ aliphatic that is optionally substituted with one to three, preferably one or two, substituents selected from the group consisting of -fluoro, —OH, —O(C₁₋₃ aliphatic), —CN, —CO₂H, —CO₂(C₁₋₃ aliphatic), —NH₂, —NH(C₁₋₃ aliphatic), —N(C₁₋₃ aliphatic)₂, and optionally substituted C₆₋₁₀ aryl. In some embodiments, R⁶ is hydrogen or an unsubstituted C₁₋₃ aliphatic.

In some embodiments, the invention relates to a compound of formula (I), wherein W is —C(O)N(R⁹)₂ or —N(R⁶)—C(O)—N(R⁹)₂, and —N(R⁹)₂ is an optionally substituted nitrogen-containing heterocyclyl. In some such embodiments, —N(R⁹)₂ is an optionally substituted morpholinyl, piperidinyl, piperazinyl, thiomorpholinyl, or pyrrolidinyl. In certain preferred embodiments, —N(R⁹)₂ is morpholinyl or 4-methylpiperazinyl.

In some other embodiments, the invention relates to a compound of formula (I), wherein W is —N(R⁶)—C(O)—R¹⁰, and R¹⁰ is a C₁₋₆ aliphatic, which optionally is substituted by 1 to 3 groups independently selected from -fluoro, —OR, CN, —CO₂R⁷, —N(R⁷)₂, —NH—C(═NH)—NH₂, or an optionally substituted aryl, cycloaliphatic, heteroaryl, or heterocyclyl group.

In certain such embodiments, W has the formula —NH—C(O)—(CF₂)ₘ—CH(N(R¹³)₂)—(CH₂)ₙ—Y¹, —NH—C(O)—CH(CH—N(R¹³)₂)—(CH₂)ₙ—Y¹, or —NH—C(O)—(CH₂)ₘ—Y¹, where R¹³ is hydrogen or C₁₋₃ aliphatic, m is 0 or 1, n is 0-3, and Y¹ is an optionally substituted aryl, cycloaliphatic, heteroaryl, or heterocyclyl group. In some embodiments, Y¹ is a C₆₋₁₀ aryl, which is optionally substituted by one to three substituents independently selected from the group consisting of -halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, and optionally substituted C₁₋₈ aliphatic, aryl, aralkyl, heterocyclyl, heterocyclylalkyl, heteroaryl, and heteroaralkyl groups.

In certain other embodiments, W has the formula —NH—C(O)—(CH₂)ₘ—CH(N(R¹³)₂)—Y², where R¹³ is hydrogen or C₁₋₃ aliphatic, m is 0 or 1, and Y² is a C₁₋₆ aliphatic, which optionally is substituted by one or two groups selected from the group consisting of -fluoro, —OR, —CN, —CO₂R⁷, —N(R⁷)₂, —NH—C(═NH)—NH₂, and —NH—C(═NH)—R.

The term “aliphatic” as used herein means a straight-chain, branched or cyclic hydrocarbon which is completely saturated or which contains one or more units of unsaturation, but which is not aromatic. When straight chained or branched, an aliphatic group is typically C₁₋₈, more typically C₁₋₆ or C₁₋₄; when cyclic, an aliphatic group is typically C₃₋₁₀, more typically C₃₋₇. For example, suitable aliphatic groups include substituted or unsubstituted linear, branched or cyclic alkyl, alkenyl, alkynyl groups and hybrids thereof, such as (cycloalkyl)alkyl, (cycloalkenyl)alkyl or (cycloalkyl)alkenyl. The terms “alkyl”, “alkoxy”, “hydroxyalkyl”, “alkoxyalkyl”, and “alkoxycarbonyl”, used alone or as part of a larger moiety include both straight and branched saturated chains containing one to eight carbon atoms. The terms “alkenyl” and alkynyl” used alone or as part of a larger moiety shall include both straight and branched chains containing two to eight carbon atoms and one or more double or triple bonds, respectively. The term “cycloaliphatic” used alone or as part of a larger moiety shall include cyclic C₃-C₁₀ hydrocarbons which are completely saturated or which contain one or more units of unsaturation, but which are not aromatic. A “cycloalkyl” is an cyclic aliphatic group that is completely saturated.

The terms “haloaliphatic”, “haloalkyl”, “haloalkenyl” and “haloalkoxy” means aliphatic, alkyl, alkenyl or alkoxy, as the case may be, substituted with one or more halogen atoms. The term “halogen” or “halo” means F, Cl, Br or I. Unless otherwise indicated, the terms “aliphatic”, “alkyl”, “alkenyl”, and “alkoxy” include haloaliphatic, haloalkyl, haloalkenyl, and haloalkoxy groups, including, in particular, those with 1-5 fluorine atoms.

The term “heteroatom” means nitrogen, oxygen, or sulfur and includes any oxidized form of nitrogen and sulfur, and the quaternized form of any basic nitrogen. Also the term “nitrogen” includes a substitutable nitrogen of a heterocyclic ring. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl) or NR⁺ (as in N-substituted pyrrolidinyl).

The term “aryl” used alone or as part of a larger moiety as in “aralkyl”, “aralkoxy”, or “aryloxyalkyl”, refers to aromatic ring groups, typically having six to fourteen members, such as phenyl, benzyl, phenethyl, 1-napthyl, 2-naphthyl, 1-anthracyl and 2-anthracyl. The term “aryl” also refers to rings that are optionally substituted. The term “aryl” may be used interchangeably with the term “aryl ring”. “Aryl” also includes fused polycyclic aromatic ring systems in which an aromatic ring is fused to one or more rings. Examples include 1-naphthyl, 2-naphthyl, 1-anthracyl and 2-anthracyl. Also included within the scope of the term “aryl”, as it is used herein, is a group in which an aromatic ring is fused to one or more non-aromatic rings, such as in an indanyl, phenanthridinyl, or tetrahydronaphthyl, where the radical or point of attachment is on the aromatic ring.

As used herein, the terms “heterocycle”, “heterocyclyl”, or “heterocyclic radical” refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms selected from the group consisting of N, O, and S, wherein the nitrogen and sulfur heteroatoms are optionally oxidized and the nitrogen atoms are optionally quaternized. The heterocyclic ring can be attached to its pendant group at any heteroatom or carbon atom that results in a stable structure, and any of the ring atoms can be optionally substituted. Examples of such saturated or partially unsaturated heterocyclic radicals include, without limitation, tetrahydrofuranyl, tetrahydrothienyl, pyrrolidinyl, pyrrolidonyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, and morpholinyl. The terms “heterocycle”, “heterocyclyl”, and “heterocyclic radical”, as used herein, also include groups in which a non-aromatic heteroatom-containing ring is fused to one or more aromatic or non-aromatic rings, such as indolinyl, chromanyl, phenanthridinyl, or tetrahydroquinolinyl, where the radical or point of attachment is on the non-aromatic heteroatom-containing ring. The term “heterocyclylalkyl” refers to an alkyl group substituted by a heterocyclyl, wherein the alkyl and heterocyclyl portions independently are optionally substituted.

The terms “heteroaryl” and “heteroar-”, used alone or as part of a larger moiety, e.g., heteroaralkyl, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to four heteroatoms selected from the group consisting of N, O, and S. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolyl, isoindolyl, benzothienyl, benzofuranyl, dibenzofuranyl, indazolyl, benzimidazolyl, benzthiazolyl, purinyl, quinolyl, isoquinolyl, cinnolinyl, phthalazinyl, quinoxalinyl, naphthyridinyl, pteridinyl, carbazolyl, acridinyl, and phenazinyl. The terms “heteroaryl” and “heteroar-”, as used herein, also include groups in which a heteroaromatic ring is fused to one or more nonaromatic rings, where the radical or point of attachment is on the heteroaromatic ring. Nonlimiting examples include tetrahydroquinolinyl, tetrahydroisoquinolinyl, and pyrido[3,4-d]pyrimidinyl. The term “heteroaryl” may be used interchangeably with the term “heteroaryl ring” or the term “heteroaromatic”, any of which terms include rings that are optionally substituted. The term “heteroaralkyl” refers to an alkyl group substituted by a heteroaryl, wherein the alkyl and heteroaryl portions independently are optionally substituted.

An “aralkyl”, “heteroaralkyl” or “heterocyclylalkyl” is an alkyl group, typically a C₁₋₈ alkyl group, substituted with an aryl (preferably phenyl), heteroaryl, or heterocyclyl group, respectively.

An “alkylidene chain” is a polymethylene group, i.e., —(CH₂)ₙ—, wherein n is a positive integer. Preferably, n is an integer from 1 to 6, more preferably from 2 to 4 and more preferably from 2 to 3. A “substituted alkylidene chain” is an alkylidene in which one or more methylene hydrogen atoms is replaced with a substituent. Suitable substituents are as described below for a substituted aliphatic group.

An alkylidene chain can be optionally interrupted by a functional group. An alkylidene chain is interrupted by a functional group when one of the internal methylenes of the alkylidene chain is replaced with the functional group. Examples of suitable “interrupting functional groups” include —O—, —S—, —N(R⁵)—, —S(O)—, —SO₂—, —C(O)—, —OC(O)—, —N(R⁵)C(O)—, —C(O)N(R⁵)—, —SO₂N(R⁵)—, and —N(R⁵)SO₂—. R⁵ is as described above. Examples of alkylidene chains which have been “interrupted” with —O— include —CH₂O(CH₂)—, —CH₂O(CH₂)₂—, —CH₂O(CH₂)₃—, —CH₂O(CF)₄—, —(CH₂)₂O(CH₂)—, —(CH₂)₂O(CH₂)₂—, —(CH₂)₂O(CH₂)₃—, —(CH₂)₃O(CH₂)—, —(CH₂)₃O(CH₂)₂—, and —(CH₂)₄O(CH₂)—. Other examples of alkylidene chains which have been “interrupted” with functional groups include —CH₂M(CH₂)—, —CH₂M(CH₂)₂—, —CH₂M(CH₂)₃—, —CH₂M(CH₂)₂—, —(CH₂)₂M(CH₂)—, —(CH₂)₂M(CH₂)₂—, —(CH₂)₂M(CH₂)₃—, —(CH₂)₃M(CH₂)—, —(CH)₃M(CH₂)₂—, and —(CH₂)₄M(CH₂)— and wherein M is one of the “interrupting” functional groups listed above.

An aryl (including aralkyl, aralkoxy, aryloxyalkyl and the like) or heteroaryl (including heteroaralkyl and heteroarylalkoxy and the like) group may contain one or more substituents. Examples of suitable substituents on an unsaturated carbon atom of an aryl, heteroaryl, aralkyl, or heteroaralkyl group include a halogen —R°, —OR°, —SR°, 1,2-methylene-dioxy, 1,2-ethylenedioxy, protected OH (such as acyloxy), phenyl (Ph), substituted Ph, —O(Ph), substituted —O(Ph), —CH₂(Ph), substituted —CH₂(Ph), —CH₂CH₄(Ph), substituted —CH₂CH₂(Ph), —NO₂, —CN, —N(R′)₂, —NR′CO₂R°, NR′C(O)R°, —NR′NR′C(O)R°, —N(R′)C(O)N(R′)₂, —NR′NR′C(O)N(R′)₂, —NR′NR′CO₂R°, —C(O)C(O)R°, —C(O)CH₂C(O)R°, —CO₂R°, —C(O)R°, —C(O)N(R°)₂, —OC(O)N(R°)₂, —S(O)₂R°, —SO₂N(R′)₂, —S(O)R°, —NR′SO₂N(R′)₂, —NR′SO₂R°, —C(═S)N(R′)₂, —(CH₂)_{y}NR′)₂, —C(═NH)—N(R′)₂, —(CH₂)_{y}NHC(O)R°, —(CH₂)_{y}NHC(O)CH(L-R°)(R°). R′ is R°, —CO₂R°, —SO₂R° or —C(O)R° and preferably hydrogen, C₁₋₆ aliphatic, CO₂R°, SO₂R° or C(O)R°. R° is hydrogen or substituted or unsubstituted aliphatic, aryl, aralkyl, heterocyclyl, heterocyclylalkyl or heteroaryl and preferably hydrogen, C₁₋₆ alkyl, phenyl (Ph), —CH₂ (Ph), aralkyl, heterocyclyl, heterocyclylalkyl or heteroaryl; y is 0-6; and L is a linker group. Examples of substituents on the aliphatic group or the phenyl ring of R° include amino, alkylamino, dialkylamino, aminocarbonyl, halogen, alkyl, aminoalkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl. Preferred substituents for Ring A are substituents represented by R⁴. Preferred substituents for Ring D are C₁₋₄ aliphatic or haloaliphatic, —OR⁷, —SR⁷, —C(O)R⁷, —CO₂R⁷, —SO₂R⁷, —CN, —C(O)N(R⁷)₂, —N(R⁷)C(O)(C₁₋₂alkyl), or —N(R⁷)₂, wherein R⁷ is as defined above. Certain particularly preferred substituents for Ring D are —CN, —COOR⁷ and —CON(R⁷)₂ at the position para to the carbon bonded to the urea nitrogen. These substituents are even more preferred when Ring D is pyridine.

An aliphatic group or a heterocycle may contain one or more substituents. Examples of suitable substituents on the saturated carbon of an aliphatic group of a heterocycle include those listed above for the unsaturated carbon of an aryl or heteroaryl group and the following: ═O, ═S, ═NNHR*, ═NN(R*)₂, ═NNHC(O)R*, ═NNHCO₂ (alkyl), ═NNHSO₂ (alkyl), or ═NR*. Each R* is independently selected from hydrogen, an unsubstituted aliphatic group or a substituted aliphatic group. Examples of substituents on the aliphatic group represented by R* include amino, alkylamino, dialkylamino, aminocarbonyl, halogen, alkyl, alkylaminocarbonyl, dialkylaminocarbonyl, alkylaminocarbonyloxy, dialkylaminocarbonyloxy, alkoxy, nitro, cyano, carboxy, alkoxycarbonyl, alkylcarbonyl, hydroxy, haloalkoxy, or haloalkyl.

Suitable substituents on the nitrogen atom of a nonaromatic heterocycle include —R⁺, —N(R⁺)₂, —C(O)R⁺, —CO₂R⁺, —C(O)C(O)R⁺, —C(O)CH₂C(O)R⁺, —SO₂R⁺, —SO₂N(R⁺)₂, —C(═S)N(R⁺)₂, —C(═NH)—N(R⁺)₂, and —NR⁺SO₂R⁺; wherein R⁺ is hydrogen, an aliphatic group, a substituted aliphatic group, phenyl (Ph), substituted Ph, —O(Ph), substituted —O(Ph), CH₂(Ph), or an unsubstituted heteroaryl or heterocyclic ring. Examples of substituents on the aliphatic group or the phenyl ring represented by R+include amino, alkylamino, dialkylamino, aminocarbonyl, halogen, alkyl, alkylaminocarbonyl, dialkylaminocarbonyloxy, alkoxy, nitro, cyano, carboxy, alkoxycarbonyl, alkylcarbonyl, hydroxy, haloalkoxy, or haloalkyl.

As noted above, Ring A of formula (I) is optionally substituted with one or more groups R⁴ and Ring D of formula (I) is optionally substituted with C₁₋₄ aliphatic, C₁₋₄ haloaliphatic, —OR⁷, —SR⁷, —C(O)R, —CO₂R⁷, —SO₂R, —CN, —C(O)N(R⁷)₂, —N(R⁷)C(O)R or —N(R⁷)₂, and is optionally fused to a six membered aromatic ring (preferably phenyl) or cyclohexyl ring. R, R⁴ and R⁷ are as described above, and the fused six membered aromatic or cyclohexyl ring is optionally substituted.

Representative examples of compounds of formula (I) are shown in Table 1.

TABLE 1
Examples of Compounds of Formula (I)

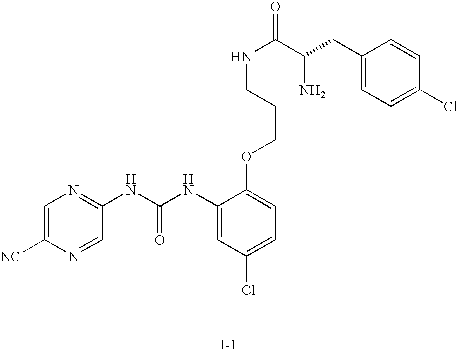

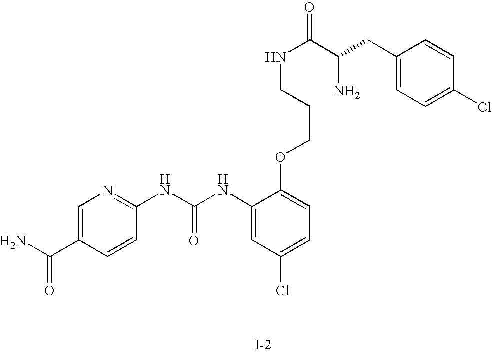

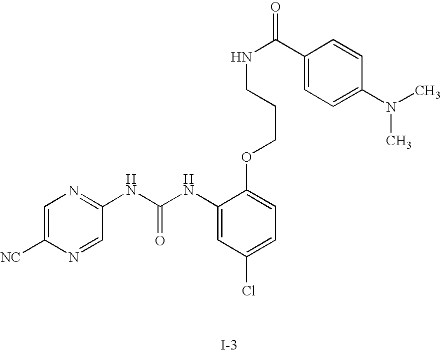

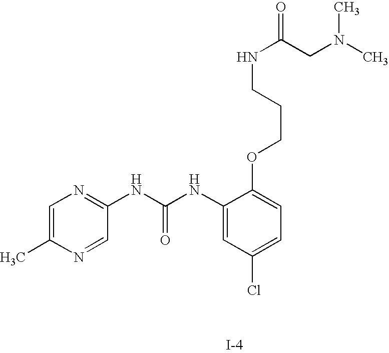

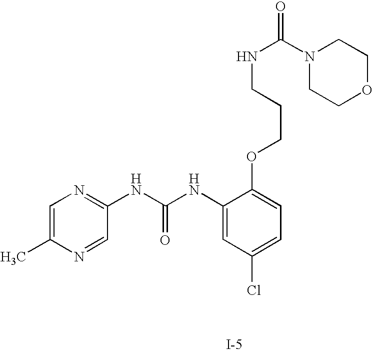

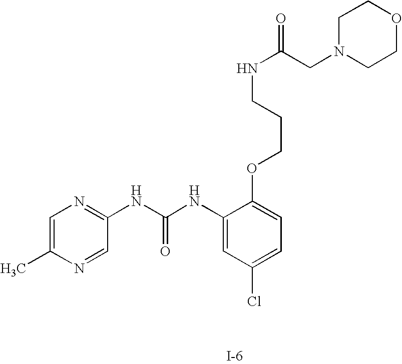

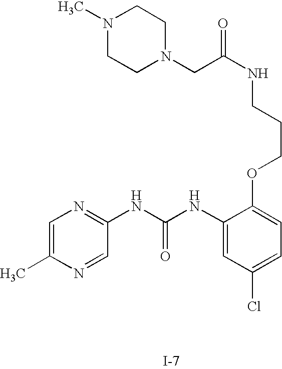

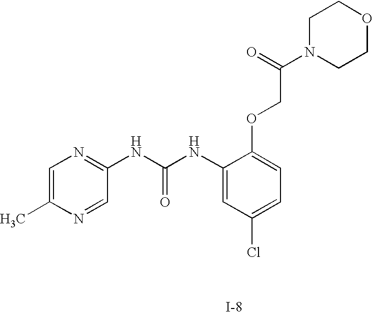

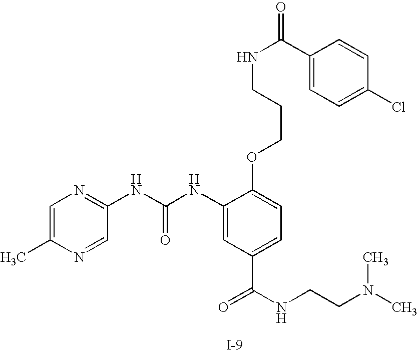

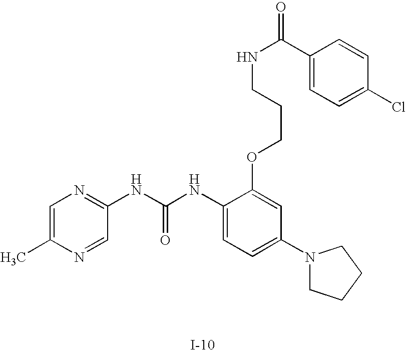

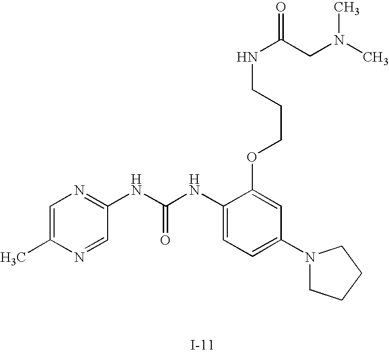

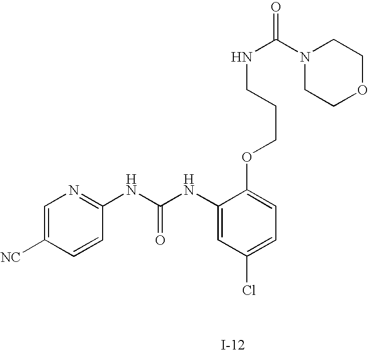

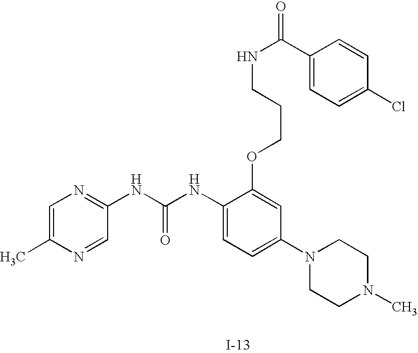

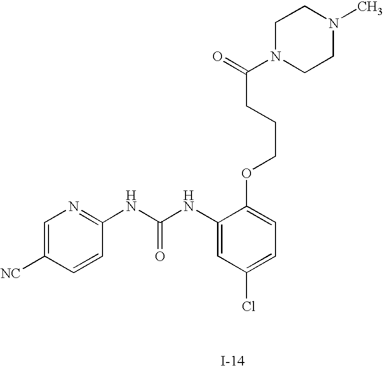

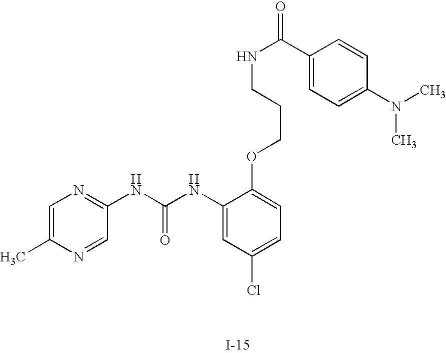

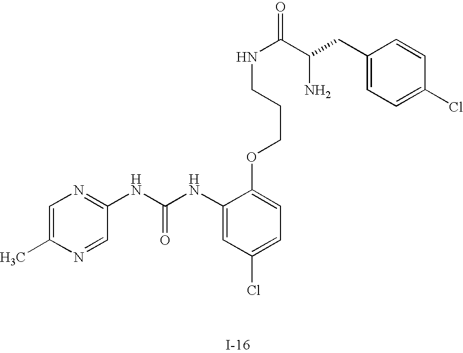

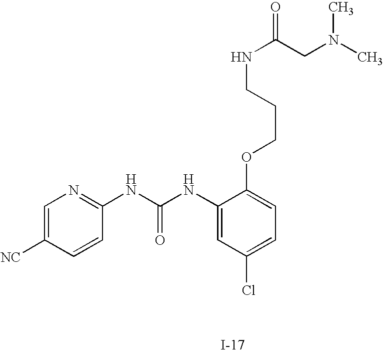

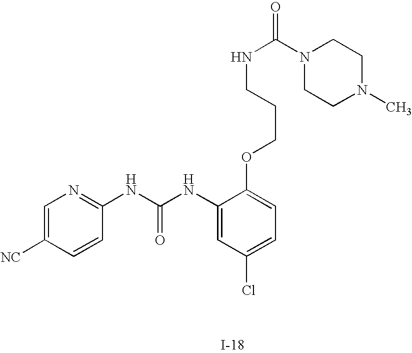

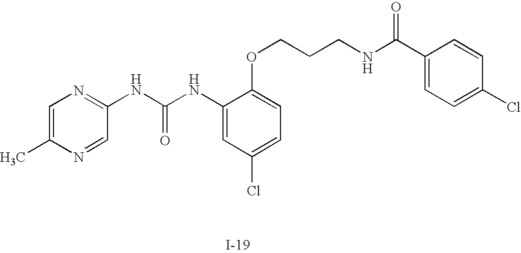

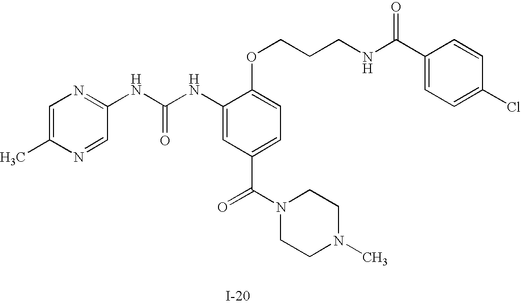

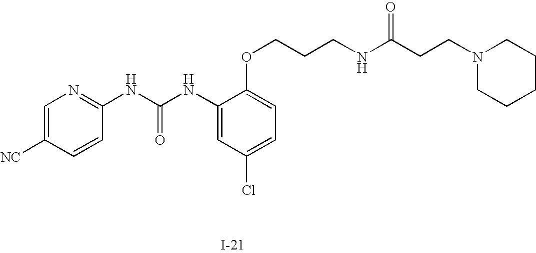

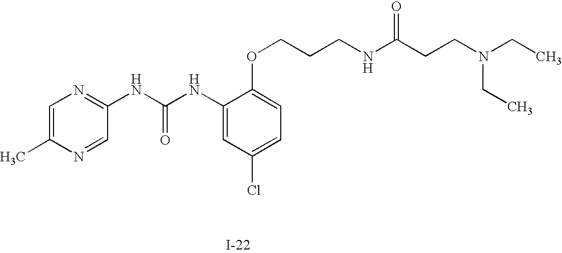

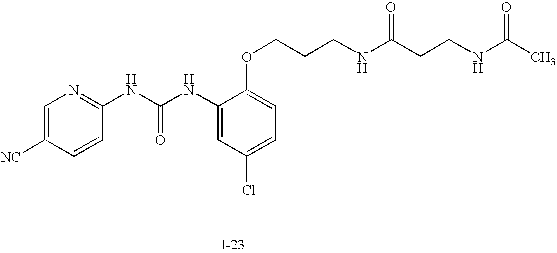

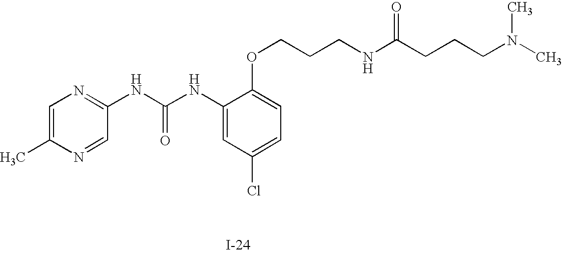

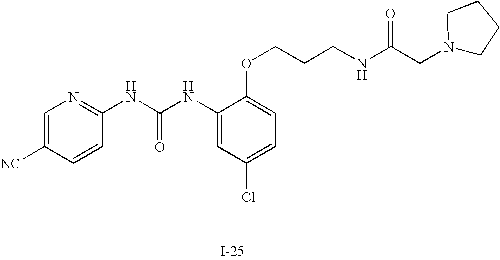

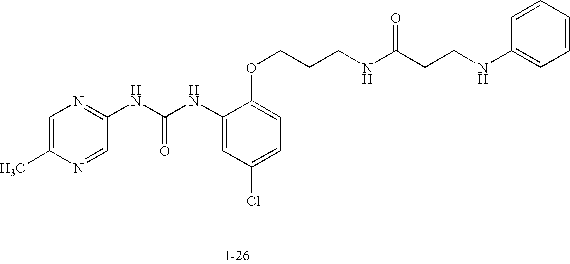

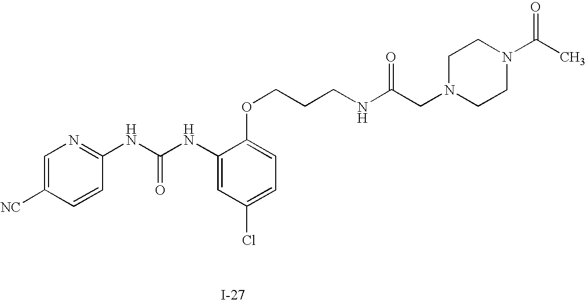

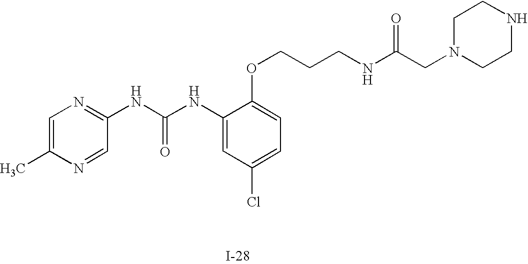

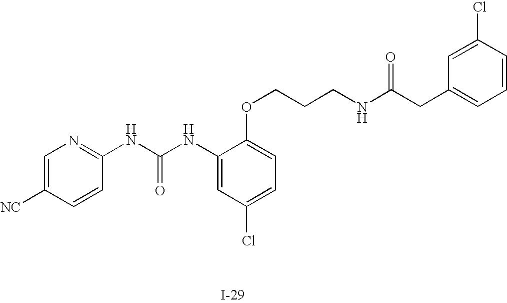

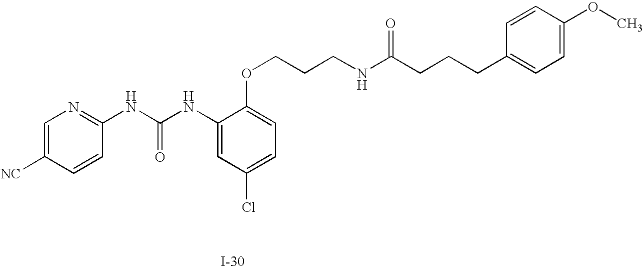

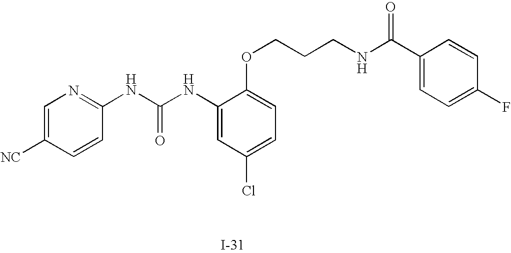

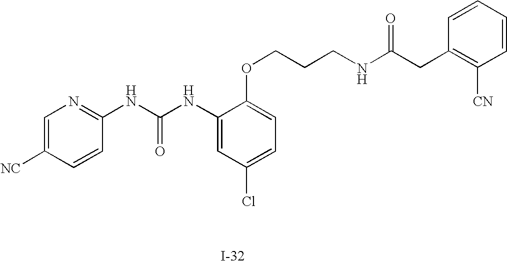

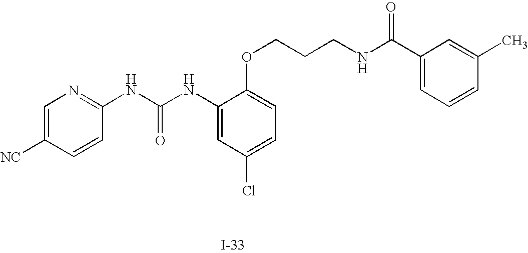

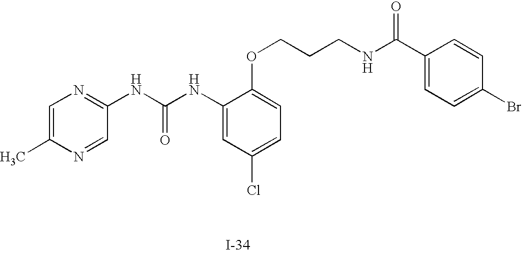

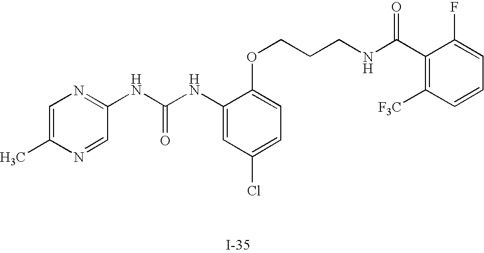

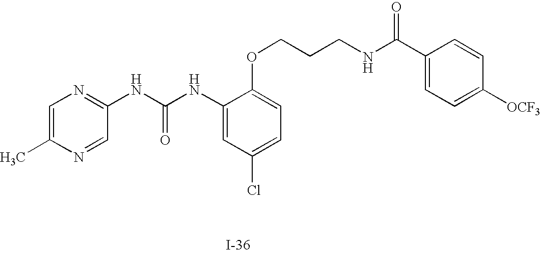

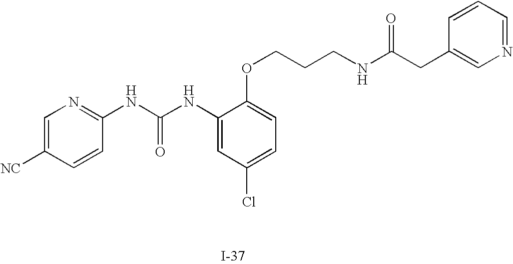

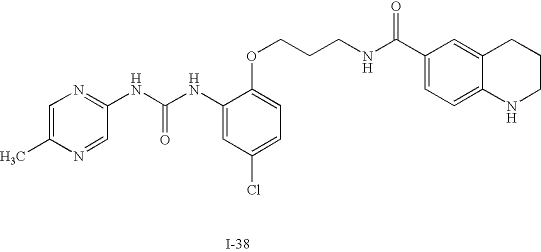

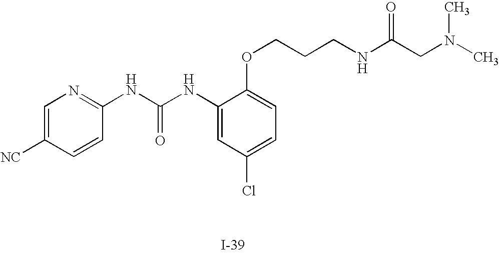

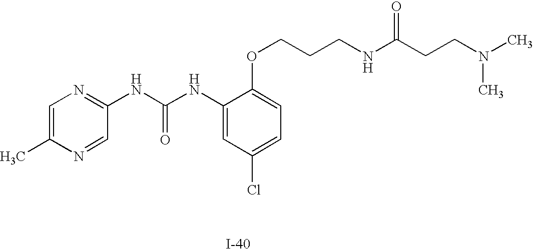

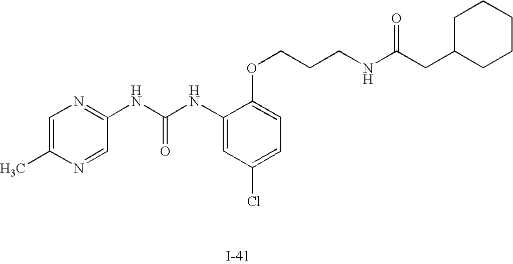

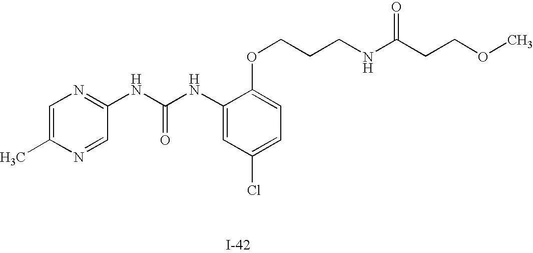

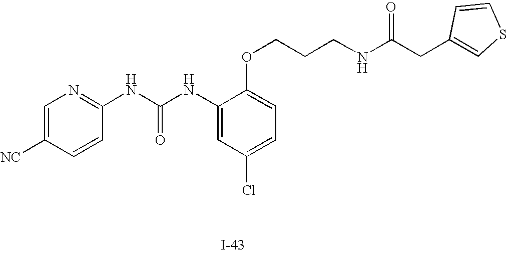

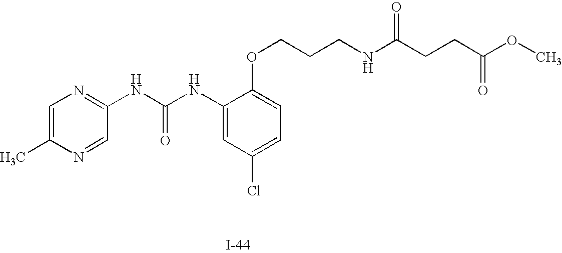

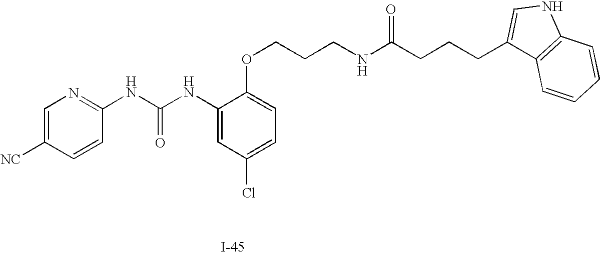

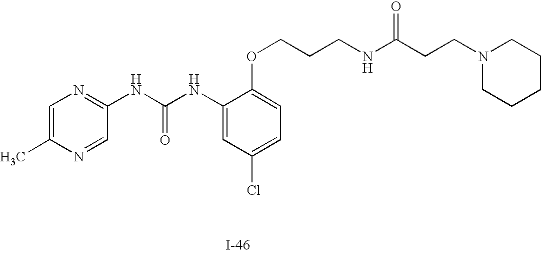

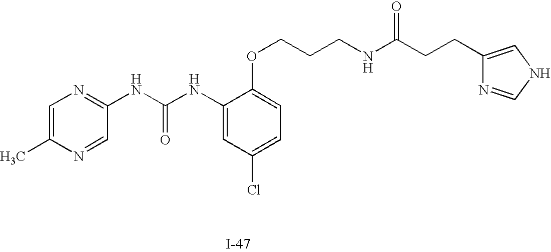

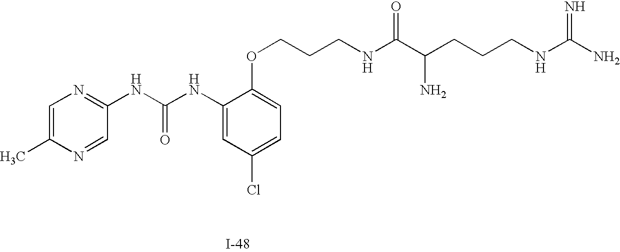

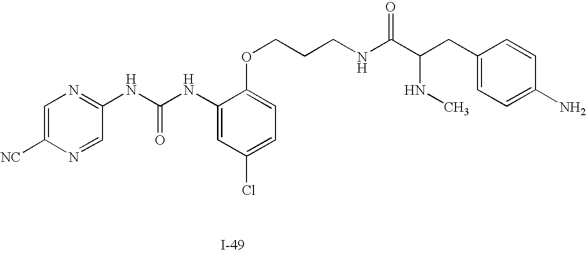

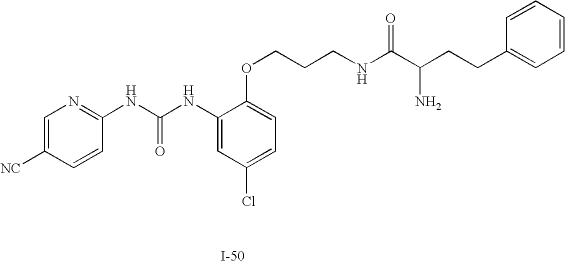

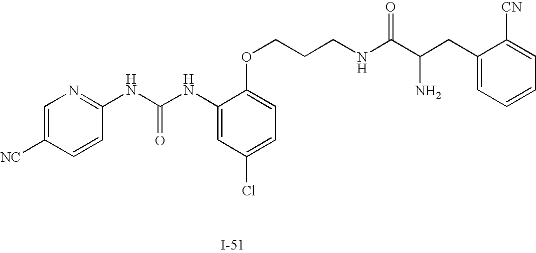

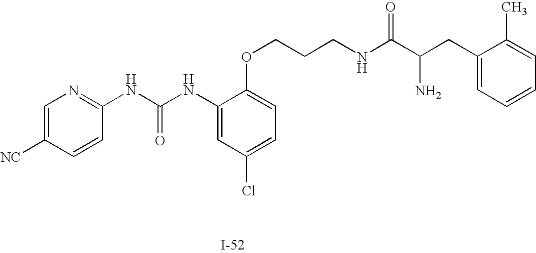

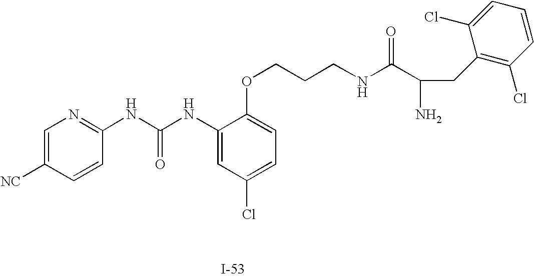

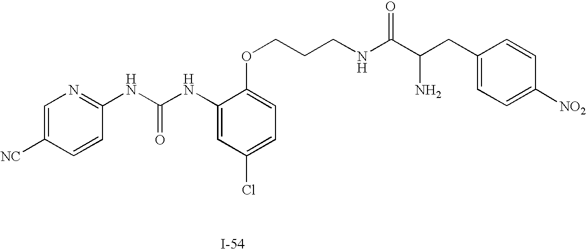

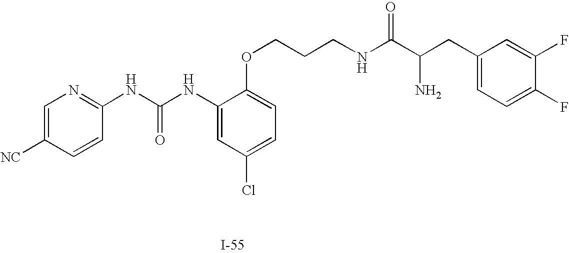

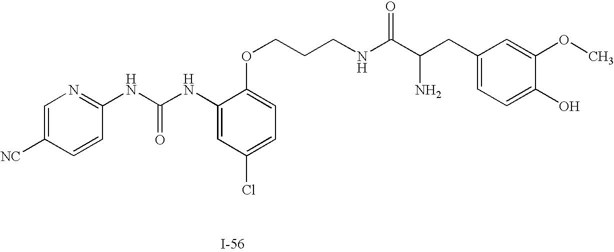

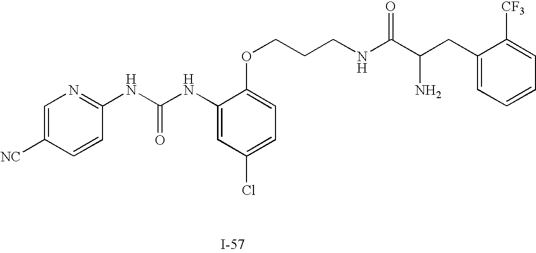

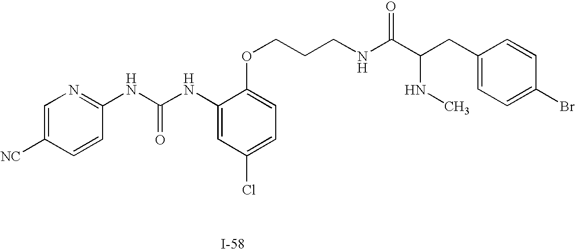

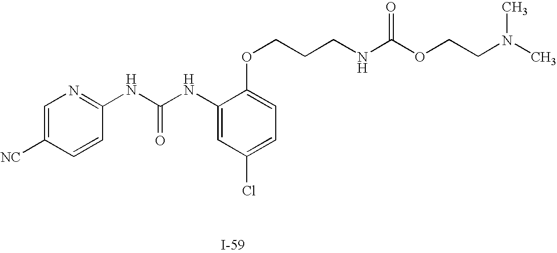

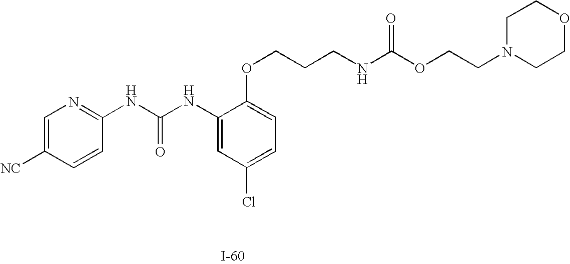

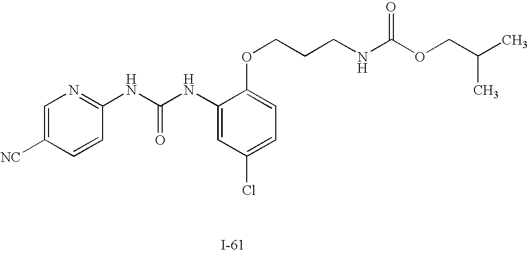

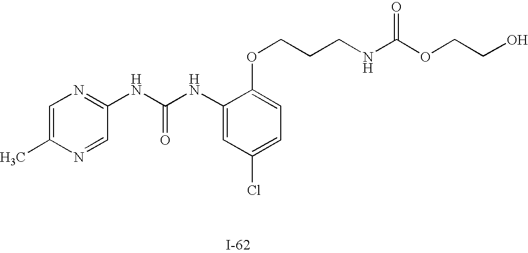

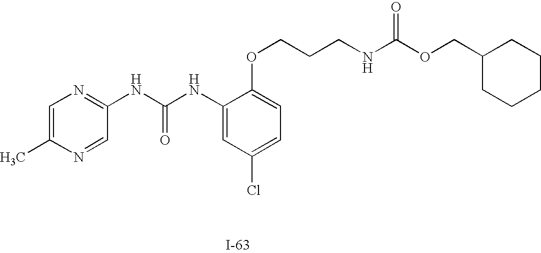

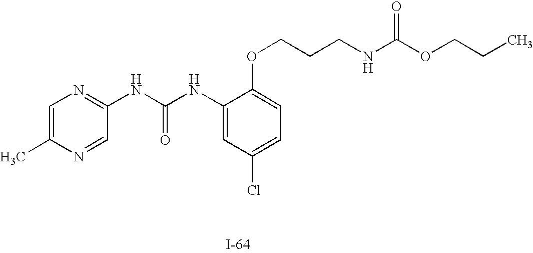

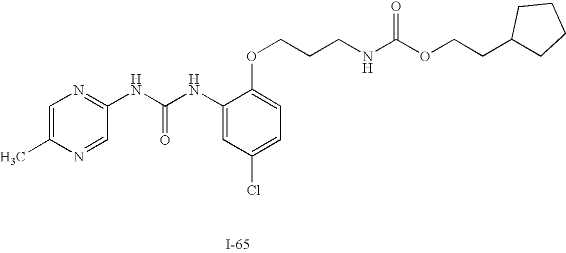

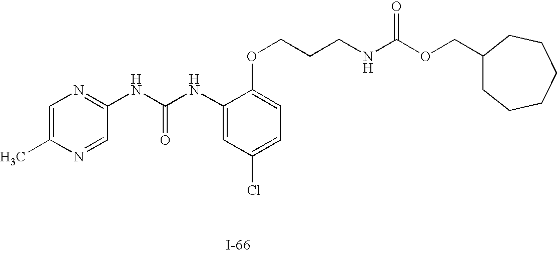

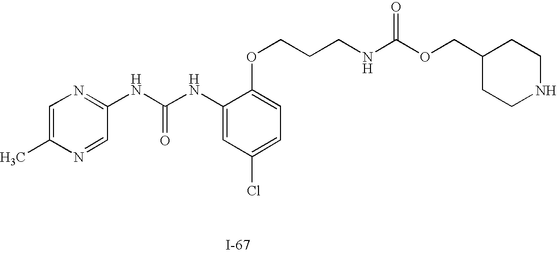

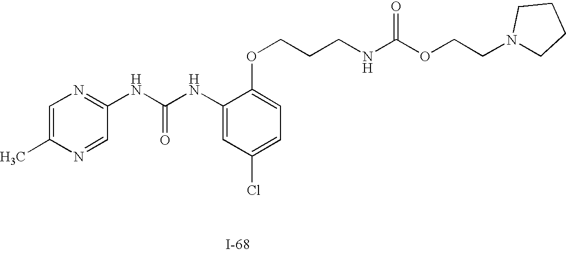

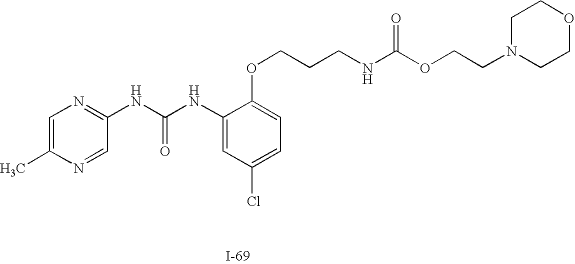

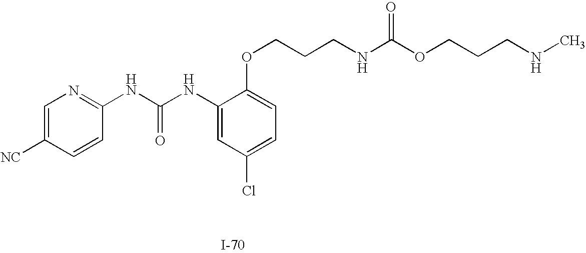

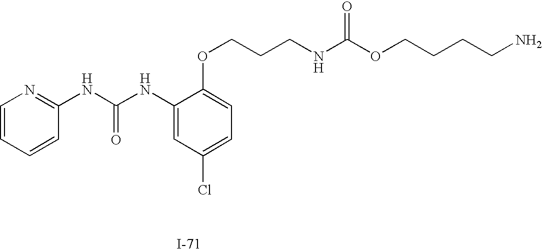

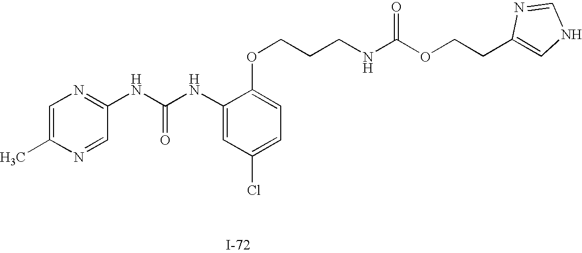

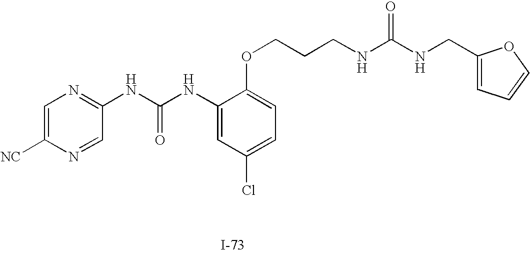

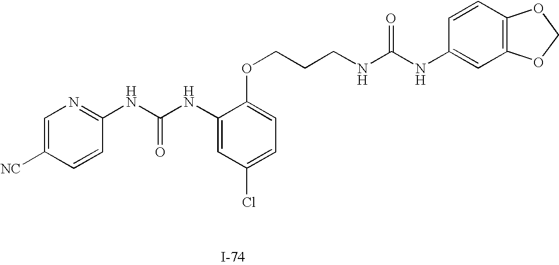

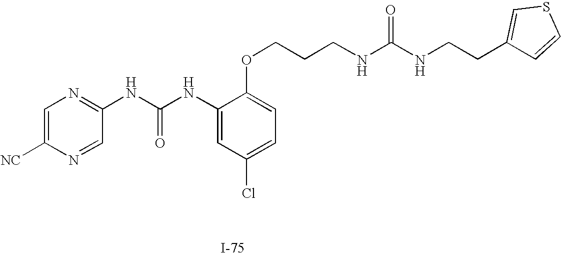

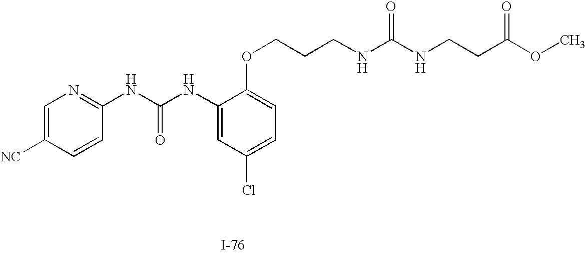

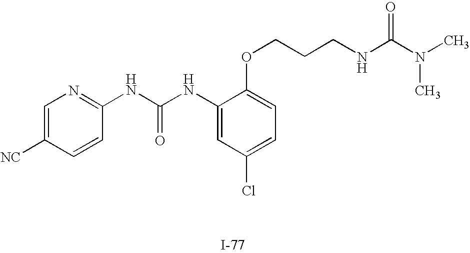

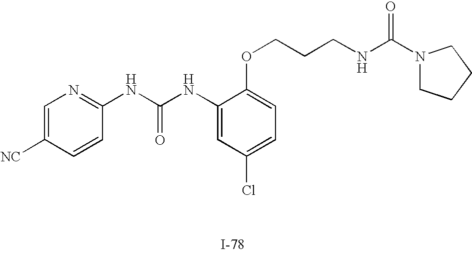

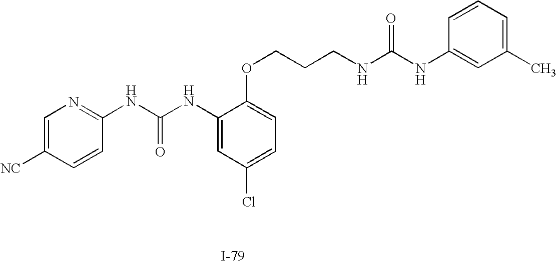

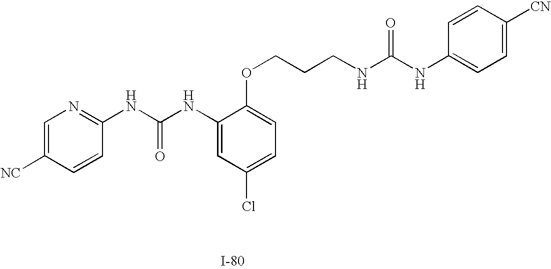

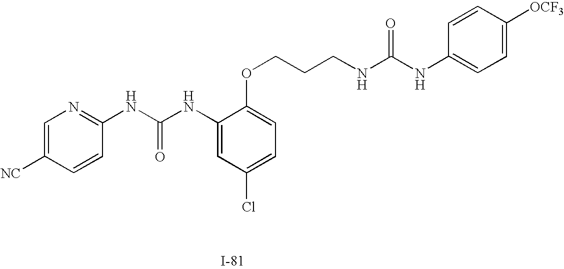

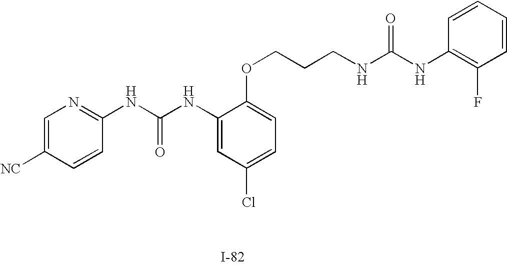

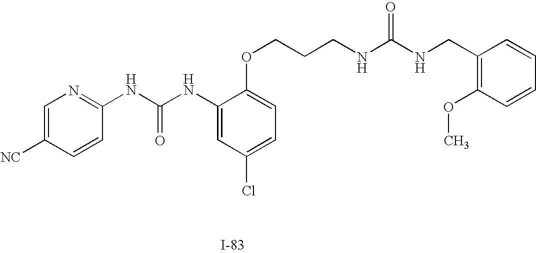

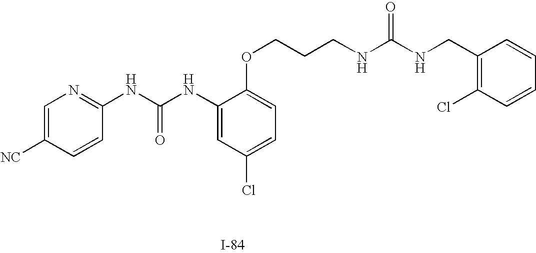

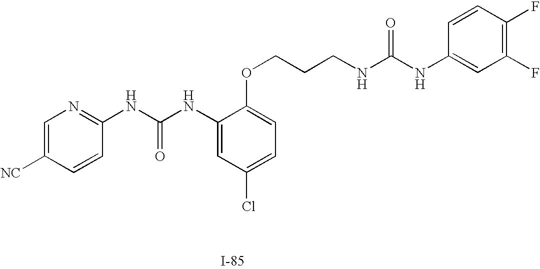

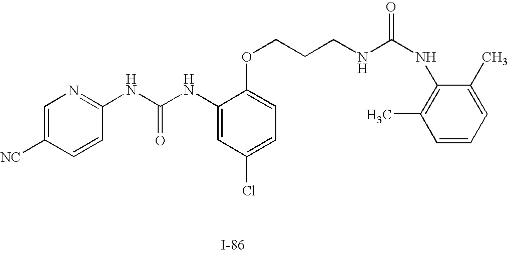

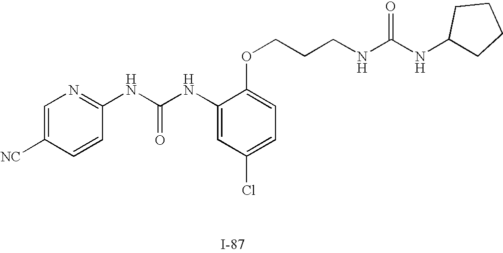

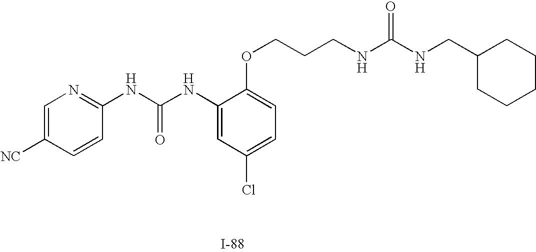

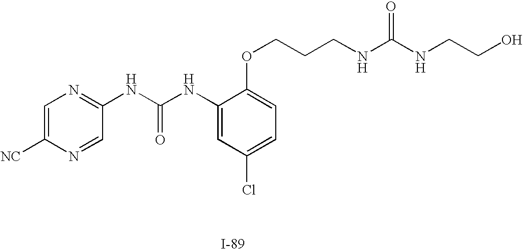

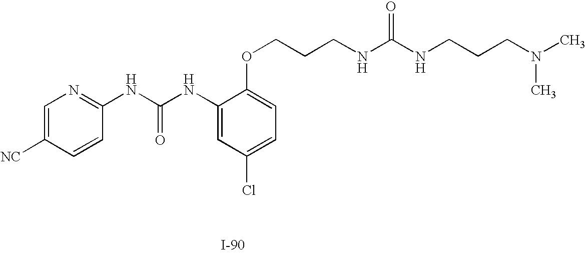

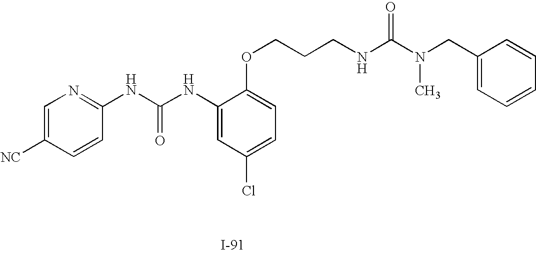

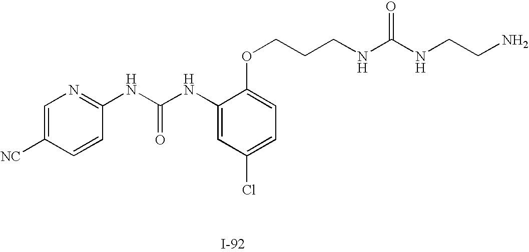

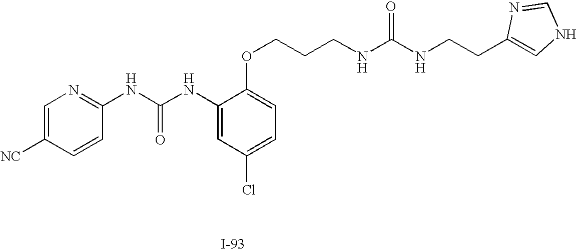

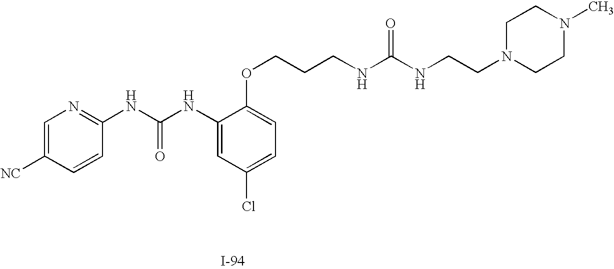

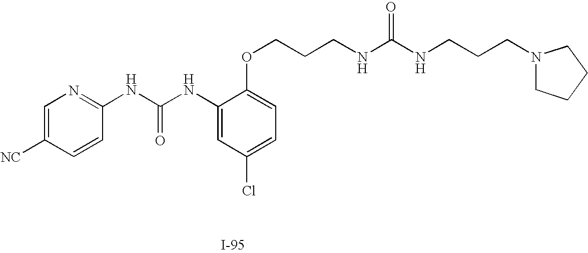

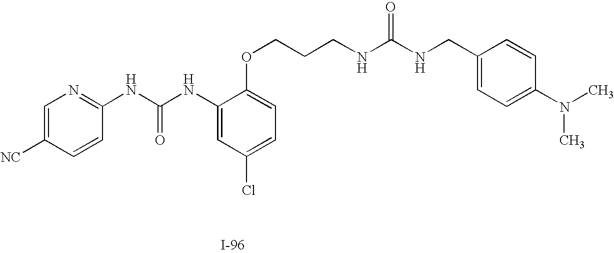

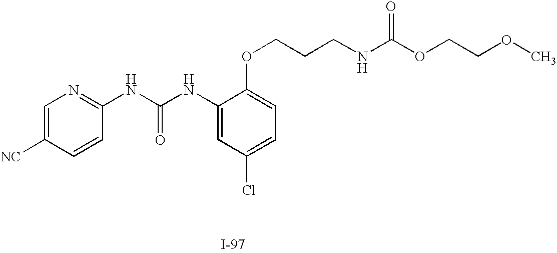

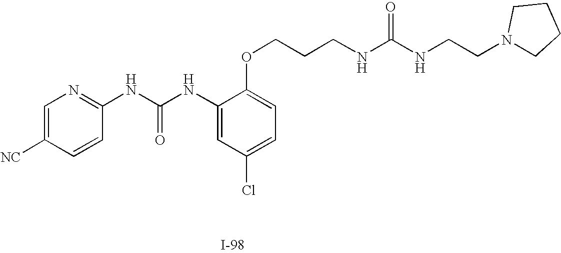

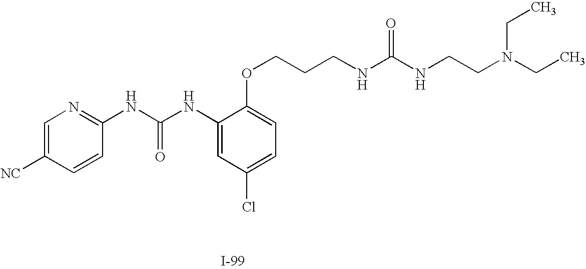

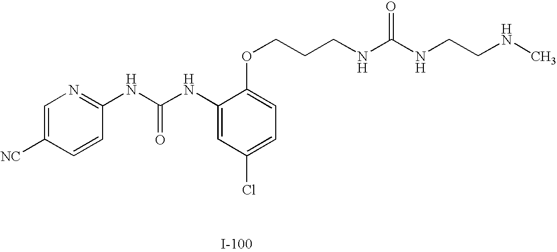

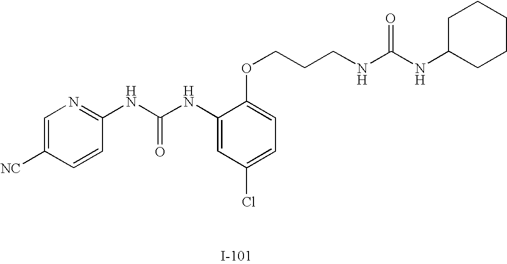

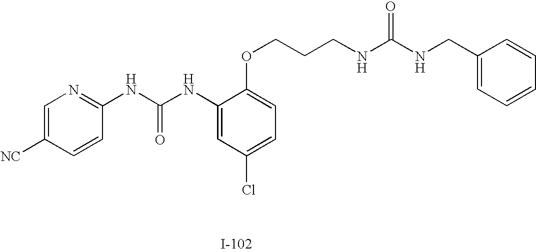

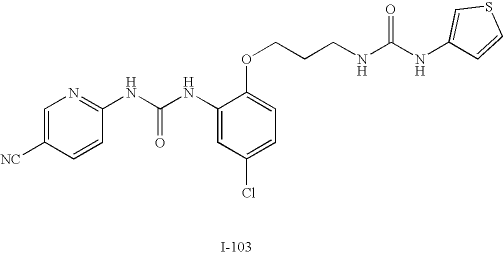

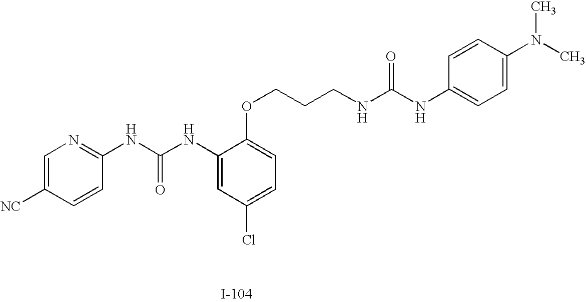

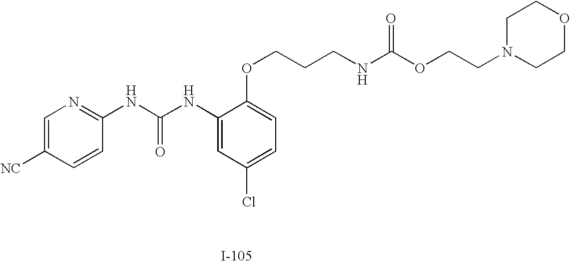

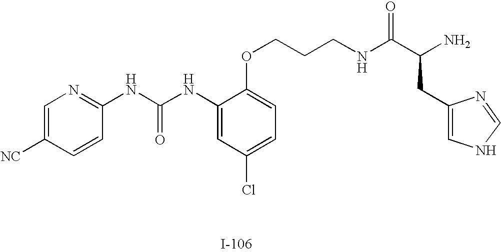

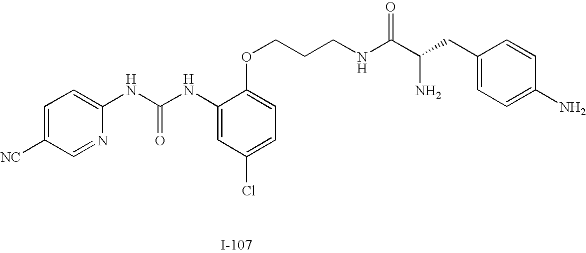

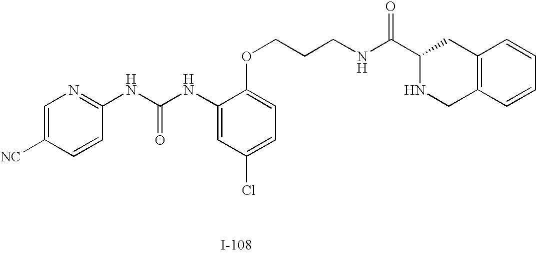

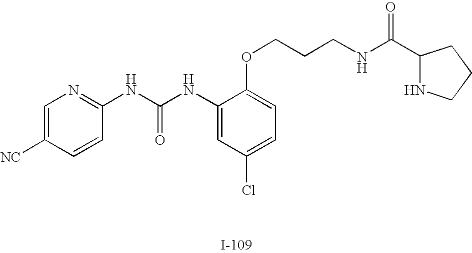

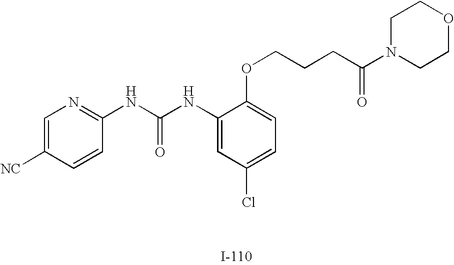

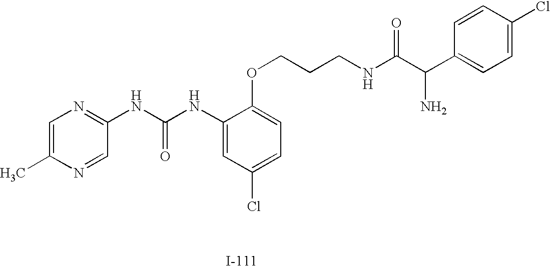

The compounds in Table 1 above also may be identified by the following chemical names:
I-1: 2-Amino-N-(3-{4-chloro-2-[3-(5-cyano-pyridin-2-yl)-ureido]-phenoxy}-propyl)-3-(4-chloro-phenyl)-propionamide
I-2: 6-[3-(2-{3-[2-Amino-3-(4-chloro-phenyl)-propionylamino]-propoxy}-5-chloro-phenyl)-ureido]-nicotinamide
I-3: 2-Amino-N-(3-{4-chloro-2-[3-(5-methyl-pyrazin-2-yl)-ureido]-phenoxy}-propyl)-2-(4-chloro-phenyl)-acetamide
I-4: N-(3-{4-Chloro-2-[3-(5-methyl-pyrazin-2-yl)-ureido]-phenoxy}-propyl)-2-dimethylamino-acetamide
I-5: Morpholine-4-carboxylic acid (3-{4-chloro-2-[3-(5-methyl-pyrazin-2-yl)-ureido]-phenoxy}-propyl)-amide
I-6: N-(3-{4-Chloro-2-[3-(5-methyl-pyrazin-2-yl)-ureido]-phenoxy}-propyl)-2-morpholin-4-yl-acetamide
I-7: N-(3-{4-Chloro-2-[3-(5-methyl-pyrazin-2-yl)-ureido]-phenoxy}-propyl)-2-(4-methyl-piperazin-1-yl)-acetamide
I-8: 1-[5-Chloro-2-(2-morpholin-4-yl-2-oxo-ethoxy)-phenyl]-3-(5-methyl-pyrazin-2-yl)-urea
I-9: 4-[3-(4-Chloro-benzoylamino)-propoxy]-N-(2-dimethylamino-ethyl)-3-[3-(5-methyl-pyrazin-2-yl)-ureido]-benzamide
I-10: 4-Chloro-N-(3-{2-[3-(5-methyl-pyrazin-2-yl)-ureido]-5-pyrrolidin-1-yl-phenoxy}-propyl)-benzamide
I-11: 2-Dimethylamino-N-(3-{2-[3-(5-methyl-pyrazin-2-yl)-ureido]-5-pyrrolidin-1-yl-phenoxy}-propyl)-acetamide
I-12: Morpholine-4-carboxylic acid (3-{4-chloro-2-[3-(5-cyano-pyridin-2-yl)-ureido]-phenoxy}-propyl)-amide
I-13: 4-Chloro-N-(3-{5-(4-methyl-piperazin-1-yl)-2-[3-(5-methyl-pyrazin-2-yl)-ureido]-phenoxy}-propyl)-benzamide
I-14: 1-{5-Chloro-2-[4-(4-methyl-piperazin-1-yl)-4-oxo-butoxy]-phenyl}-3-(5-cyano-pyridin-2-yl)-urea
I-15: N-(3-{4-Chloro-2-[3-(5-methyl-pyrazin-2-yl)-ureido]-phenoxy}-propyl)-4-dimethylamino-benzaminde
I-16: 2-Amino-N-(3-{4-chloro-2-[3-(5-methyl-pyrazin-2-yl)-ureido]-phenoxy}-propyl)-3-(4-chloro-phenyl)-propionamide
I-17: N-(3-{4-Chloro-2-[3-(5-cyano-pyridin-2-yl)-ureido]-phenoxy}-propyl)-2-dimethylamino-acetamide
I-18: 4-Methyl-piperazine-1-carboxylic acid (3-{4-chloro-2-[3-(5-cyano-pyridin-2-yl)-ureido]-phenoxy}-propyl)-amide
I-19: 4-Chloro-N-(3-{4-chloro-2-[3-(5-methyl-pyrazin-2-yl)-ureido]-phenoxy}-propyl)-benzamide
I-20: 4-Chloro-N-(3-{4-(4-methyl-piperazine-1-carbonyl)-2-[3-(5-methyl-pyrazin-2-yl)-ureido]-phenoxy}-propyl)-benzamide
I-21: N-(3-{4-Chloro-2-[3-(5-cyano-pyridin-2-yl)-ureido]-phenoxy}-propyl)-3-piperidin-1-yl-propionamide
I-22: N-(3-{4-Chloro-2-[3-(5-methyl-pyrazin-2-yl)-ureido]-phenoxy}-propyl)-3-diethylamino-propionamide
I-23: 3-Acetylamino-N-(3-{4-chloro-2-[3-(5-cyano-pyridin-2-yl)-ureido]-phenoxy}-propyl)-propionamide
I-24: N-(3-{4-Chloro-2-[3-(5-methyl-pyrazin-2-yl)-ureido]-phenoxy}-propyl)-4-dimethylamino-butyramide
I-25: N-(3-{4-Chloro-2-[3-(5-cyano-pyridin-2-yl)-ureido]-phenoxy}-propyl)-2-pyrrolidin-1-yl-acetamide
I-26: N-(3-{4-Chloro-2-[3-(5-methyl-pyrazin-2-yl)-ureido]-phenoxy}-propyl)-3-phenylamino-propionamide
I-27: 2-(4-Acetyl-piperazin-1-yl)-N-(3-{4-chloro-2-[3-(5-cyano-pyridin-2-yl)-ureido]-phenoxy}-propyl)-acetamide
I-28: N-(3-{4-Chloro-2-[3-(5-methyl-pyrazin-2-yl)-ureido]-phenoxy}-propyl)-2-piperazin-1-yl-acetamide
I-29: N-(3-{4-Chloro-2-[3-(5-cyano-pyridin-2-yl)-ureido]-phenoxy}-propyl)-2-(3-chloro-phenyl)-acetamide
I-30: N-(3-{4-Chloro-2-[3-(5-cyano-pyridin-2-yl)-ureido]-phenoxy}-propyl)-4-(4-methoxy-phenyl)-butyramide
I-31: N-(3-{4-Chloro-2-[3-(5-cyano-pyridin-2-yl)-ureido]-phenoxy}-propyl)-4-fluoro-benzamide
I-32: N-(3-{4-Chloro-2-[3-(5-cyano-pyridin-2-yl)-ureido]-phenoxy}-propyl)-2-(2-cyano-phenyl)-acetamide
I-33: N-(3-{4-Chloro-2-[3-(5-cyano-pyridin-2-yl)-ureido]-phenoxy}-propyl)-3-methyl-benzamide
I-34: 4-Bromo-N-(3-{4-chloro-2-[3-(5-methyl-pyrazin-2-yl)-ureido]-phenoxy}-propyl)-benzamide
I-35: N-(3-{4-Chloro-2-[3-(5-methyl-pyrazin-2-yl)-ureido]-phenoxy}-propyl)-2-fluoro-6-trifluoromethyl-benzamide
I-36: N-(3-{4-Chloro-2-[3-(5-methyl-pyrazin-2-yl)-ureido]-phenoxy}-propyl)-4-trifluoromethoxy-benzamide
I-37: N-(3-{4-Chloro-2-[3-(5-cyano-pyridin-2-yl)-ureido]-phenoxy}-propyl)-2-pyridin-3-yl-acetamide
I-38: 1,2,3,4-Tetrahydro-quinoline-6-carboxylic acid (3-{4-chloro-2-[3-(5-methyl-pyrazin-2-yl)-ureido]-phenoxy}-propyl)-amide
I-39: N-(3-{4-Chloro-2-[3-(5-cyano-pyridin-2-yl)-ureido]-phenoxy}-propyl)-2-dimethylamino-acetamide
I-40: N-(3-{4-Chloro-2-[3-(5-methyl-pyrazin-2-yl)-ureido]-phenoxy}-propyl)-3-dimethylamino-propionamide
I-41: N-(3-{4-Chloro-2-[3-(5-methyl-pyrazin-2-yl)-ureido]-phenoxy}-propyl)-2-cyclohexyl-acetamide
I-42: N-(3-{4-Chloro-2-[3-(5-methyl-pyrazin-2-yl)-ureido]-phenoxy}-propyl)-3-methoxy-propionamide
I-43: N-(3-{4-Chloro-2-[3-(5-cyano-pyridin-2-yl)-ureido]-phenoxy}-propyl)-2-thiophen-3-yl-acetamide
I-44: N-(3-{4-Chloro-2-[3-(5-methyl-pyrazin-2-yl)-ureido]-phenoxy}-propyl)-succinamic acid methyl ester
I-45: N-(3-{4-Chloro-2-[3-(5-cyano-pyridin-2-yl)-ureido]-phenoxy}-propyl)-4-(1H-indol-3-yl)-butyramide
I-46: N-(3-{4-Chloro-2-[3-(5-methyl-pyrazin-2-yl)-ureido]-phenoxy}-propyl)-3-piperidin-1-yl-propionamide
I-47: N-(3-{4-Chloro-2-[3-(5-methyl-pyrazin-2-yl)-ureido]-phenoxy}-propyl)-3-(1H-imidazol-4-yl)-propionamide
I-48: 2-Amino-5-guanidino-pentanoic acid (3-{4-chloro-2-[3-(5-methyl-pyrazin-2-yl)-ureido]-phenoxy}-propyl)-amide
I-49: 3-(4-Amino-phenyl)-N-(3-{4-chloro-2-[3-(5-cyano-pyridin-2-yl)-ureido]-phenoxy}-propyl)-2-methylamino-propionamide
I-50: 2-Amino-N-(3-{4-chloro-2-[3-(5-cyano-pyridin-2-yl)-ureido]-phenoxy}-propyl)-4-phenyl-butyramide
I-51: 2-Amino-N-(3-{4-chloro-2-[3-(5-cyano-pyridin-2-yl)-ureido]-phenoxy}-propyl)-3-(2-cyano-phenyl)-propionamide
I-52: 2-Amino-N-(3-{4-chloro-2-[3-(5-cyano-pyridin-2-yl)-ureido]-phenoxy}-propyl)-3-o-tolyl-propionamide
I-53: 2-Amino-N-(3-{4-chloro-2-[3-(5-cyano-pyridin-2-yl)-ureido]-phenoxy}-propyl)-3-(2,6-dichloro-phenyl)-propionamide
I-54: 2-Amino-N-(3-{4-chloro-2-[3-(5-cyano-pyridin-2-yl)-ureido]-phenoxy}-propyl)-3-(4-nitro-phenyl)-propionamide
I-55: 2-Amino-N-(3-{4-chloro-2-[3-(5-cyano-pyridin-2-yl)-ureido]-phenoxy}-propyl)-3-(3,4-difluoro-phenyl)-propionamide
I-56: 2-Amino-N-(3-{4-chloro-2-[3-(5-cyano-pyridin-2-yl)-ureido]-phenoxy}-propyl)-3-(4-hydroxy-3-methoxy-phenyl)-propionamide
I-57: 2-Amino-N-(3-{4-chloro-2-[3-(5-cyano-pyridin-2-yl)-ureido]-phenoxy}-propyl)-3-(2-trifluoromethyl-phenyl)-propionamide
I-58: 3-(4-Bromo-phenyl)-N-(3-{4-chloro-2-[3-(5-cyano-pyridin-2-yl)-ureido]-phenoxy}-propyl)-2-methylamino-propionamide
I-59: (3-{4-Chloro-2-[3-(5-cyano-pyridin-2-yl)-ureido]-phenoxy}-propyl)-carbamic acid 2-dimethylamino-ethyl ester
I-60: (3-{4-Chloro-2-[3-(5-cyano-pyridin-2-yl)-ureido]-phenoxy}-propyl)-carbamic acid 2-morpholin-4-yl-ethyl ester
I-61: (3-{4-Chloro-2-[3-(5-cyano-pyridin-2-yl)-ureido]-phenoxy}-propyl)-carbamic acid isobutyl ester
I-62: (3-{4-Chloro-2-[3-(5-methyl-pyrazin-2-yl)-ureido]-phenoxy}-propyl)-carbamic acid 2-hydroxy-ethyl ester
I-63: (3-{4-Chloro-2-[3-(5-methyl-pyrazin-2-yl)-ureido]-phenoxy}-propyl)-carbamic acid cyclohexylmethyl ester
I-64: (3-{4-Chloro-2-[3-(5-methyl-pyrazin-2-yl)-ureido]-phenoxy}-propyl)-carbamic acid propyl ester
I-65: (3-{4-Chloro-2-[3-(5-methyl-pyrazin-2-yl)-ureido]-phenoxy}-propyl)-carbamic acid 2-cyclopentyl-ethyl ester
I-66: (3-{4-Chloro-2-[3-(5-methyl-pyrazin-2-yl)-ureido]-phenoxy}-propyl)-carbamic acid cycloheptylmethyl ester
I-67: (3-{4-Chloro-2-[3-(5-methyl-pyrazin-2-yl)-ureido]-phenoxy}-propyl)-carbamic acid piperidin-4-ylmethyl ester
I-68: (3-{4-Chloro-2-[3-(5-methyl-pyrazin-2-yl)-ureido]-phenoxy}-propyl)-carbamic acid 2-pyrrolidin-1-yl-ethyl ester
I-69: (3-{4-Chloro-2-[3-(5-methyl-pyrazin-2-yl)-ureido]-phenoxy}-propyl)-carbamic acid 2-morpholin-4-yl-ethyl ester
I-70: (3-{4-Chloro-2-[3-(5-cyano-pyridin-2-yl)-ureido]-phenoxy}-propyl)-carbamic acid 3-methylamino-propyl ester
I-71: {3-[4-Chloro-2-(3-pyridin-2-yl-ureido]-phenoxy}-propyl)-carbamic acid 4-amino-butyl ester
I-72: (3-{4-Chloro-2-[3-(5-methyl-pyrazin-2-yl)-ureido]-phenoxy}-propyl)-carbamic acid 2-(1H-imidazol-4-yl)-ethyl ester
I-73: 1-{5-Chloro-2-[3-(3-furan-2-ylmethyl-ureido)-propoxy]-phenyl}-3-(5-cyano-pyridin-2-yl)-urea
I-74: 1-{2-[3-(3-Benzo[1,3]dioxol-5-yl-ureido)-propoxy]-5-chloro-phenyl}-3-(5-cyano-pyridin-2-yl)-urea
I-75: 1-(5-Chloro-2-{3-[3-(2-thiophen-3-yl-ethyl)-ureido]-propoxy}-phenyl)-3-(5-cyano-pyridin-2-yl)-urea
I-76: 3-[3-(3-{4-Chloro-2-[3-(5-cyano-pyridin-2-yl)-ureido]-phenoxy}-propyl)-ureido]-propionic acid methyl ester
I-77: 1-{5-Chloro-2-[3-(3,3-dimethyl-ureido)-propoxy]-phenyl}-3-(5-cyano-pyridin-2-yl)-urea
I-78: Pyrrolidine-1-carboxylic acid (3-{4-chloro-2-[3-(5-cyano-pyridin-2-yl)-ureido]-phenoxy}-propyl)-amide
I-79: 1-{5-Chloro-2-[3-(3-m-tolyl-ureido)-propoxy]-phenyl}-3-(5-cyano-pyridin-2-yl)-urea
I-80: 1-(5-Chloro-2-{3-[3-(4-cyano-phenyl)-ureido]-propoxy}-phenyl)-3-(5-cyano-pyridin-2-yl)-urea
I-81: 1-(5-Chloro-2-{3-[3-(4-trifluoromethoxy-phenyl)-ureido]-propoxy}-phenyl)-3-(5-cyano-pyridin-2-yl)-urea
I-82: 1-(5-Chloro-2-{3-[3-(2-fluoro-phenyl)-ureido]-propoxy}-phenyl)-3-(5-cyano-pyridin-2-yl)-urea
I-83: 1-(5-Chloro-2-{3-[3-(2-methoxy-benzyl)-ureido]-propoxy}-phenyl)-3-(5-cyano-pyridin-2-yl)-urea
I-84: 1-(5-Chloro-2-{3-[3-(2-chloro-benzyl)-ureido]-propoxy}-phenyl)-3-(5-cyano-pyridin-2-yl)-urea
I-85: 1-(5-Chloro-2-{3-[3-(3,4-difluoro-phenyl)-ureido]-propoxy}-phenyl)-3-(5-cyano-pyridin-2-yl)-urea
I-86: 1-(5-Chloro-2-{3-[3-(2,6-dimethyl-phenyl)-ureido]-propoxy}-phenyl)-3-(5-cyano-pyridin-2-yl)-urea
I-87: 1-{5-Chloro-2-[3-(3-cyclopentyl-ureido)-propoxy]-phenyl}-3-(5-cyano-pyridin-2-yl)-urea
I-88: 1-{5-Chloro-2-[3-(3-cyclohexylmethyl-ureido)-propoxy]-phenyl}-3-(5-cyano-pyridin-2-yl)-urea
I-89: 1-(5-Chloro-2-{3-[3-(2-hydroxy-ethyl)-ureido]-propoxy}-phenyl)-3-(5-cyano-pyridin-2-yl)-urea
I-90: 1-(5-Chloro-2-{3-[3-(3-dimethylamino-propyl)-ureido]-propoxy}-phenyl)-3-(5-cyano-pyridin-2-yl)-urea
I-91: 1-{2-[3-(3-Benzyl-3-methyl-ureido)-propoxy]-5-chloro-phenyl}-3-(5-cyano-pyridin-2-yl)-urea
I-92: 1-(2-{3-[3-(2-Amino-ethyl)-ureido]-propoxy}-5-chloro-phenyl)-3-(5-cyano-pyridin-2-yl)-urea
I-93: 1-[5-Chloro-2-(3-{3-[2-(1H-imidazol-4-yl)-ethyl]-ureido}-propoxy)-phenyl]-3-(5-cyano-pyridin-2-yl)-urea
I-94: 1-[5-Chloro-2-(3-{3-[2-(4-methyl-piperazin-1-yl)-ethyl]-ureido}-propoxy)-phenyl]-3-(5-cyano-pyridin-2-yl)-urea
I-95: 1-(5-Chloro-2-{3-[3-(3-pyrrolidin-1-yl-propyl)-ureido]-propoxy}-phenyl)-3-(5-cyano-pyridin-2-yl)-urea
I-96: 1-(5-Chloro-2-[3-[3-(4-dimethylamino-benzyl)-ureido]-propoxy-phenyl)-3-(5-cyano-pyridin-2-yl)-urea
I-97: (3-{4-Chloro-2-[3-(5-cyano-pyridin-2-yl)-ureido]-phenoxy}-propyl)-carbamic acid 2-methoxy-ethyl ester
I-98: 1-(5-Chloro-2-{3-[3-(2-chloro-ethyl)-ureido]-propoxy}-phenyl)-3-(5-cyano-pyridin-2-yl)-urea
I-99: 1-(5-Chloro-2-{3-[3-(2-diethylamino-ethyl)-ureido]-propoxy}-phenyl)-3-(5-cyano-pyridin-2-yl)-urea
I-100: 1-(5-Chloro-2-{3-[3-(2-methylamino-ethyl)-ureido]-propoxy}-phenyl)-3-(5-cyano-pyridin-2-yl)-urea
I-101: 1-{5-Chloro-2-[3-(3-cyclohexyl-ureido)-propoxy]-phenyl-3-(5-cyano-pyridin-2-yl)-urea
I-102: 1-{2-[3-(3-Benzyl-ureido)-propoxy]-5-chloro-phenyl-3-(5-cyano-pyridin-2-yl)-urea
I-103: 1-{5-Chloro-2-[3-(3-thiophen-3-yl-ureido)-propoxy]-phenyl-3-(5-cyano-pyridin-2-yl)-urea
I-104: 1-(5-Chloro-2-(3-[3-(4-dimethylamino-phenyl)-ureido]-propoxy-phenyl)-3-(5-cyano-pyridin-2-yl)-urea
I-105: (3-{4-Chloro-2-[3-(5-cyano-pyridin-2-yl)-ureido]-phenoxy-propyl)-carbamic acid 2-morpholin-4-yl-ethyl ester
I-106: (S)-2-Amino-N-(3-{4-chloro-2-[3-(5-cyano-pyridin-2-yl)-ureido]-phenoxy}-propyl)-3-(1H-imidazol-4-yl)-propionamide
I-107: (S)-2-Amino-3-(4-amino-phenyl)-N-(3-{4-chloro-2-[3-(5-cyano-pyridin-2-yl)-ureido]-phenoxy-propyl)-propionamide
I-108: (S)-1,2,3,4-Tetrahydro-isoquinoline-3-carboxylic acid (3-{4-chloro-2-[3-(5-cyano-pyridin-2-yl)-ureido]-phenoxy-propyl)-amide
I-109: (S)-Pyrrolidine-2-carboxylic acid (3-{4-chloro-2-[3-(5-cyano-pyridin-2-yl)-ureido]-phenoxy-propyl)-amide
I-110: 1-[5-Chloro-2-(4-morpholin-4-yl-4-oxo-butoxy)-phenyl]-3-(5-cyano-pyridin-2-yl)-urea
I-111: 2-Amino-N-(3-{4-chloro-2-[3-(5-methyl-pyrazin-2-yl)-ureido]-phenoxy}-propyl)-2-(4-chloro-phenyl)-acetamide

The invention further provides a pharmaceutical composition comprising a compound of formula (I) as defined above, or a pharmaceutically acceptable salt thereof, and a pharmaceutically acceptable carrier.

If pharmaceutically acceptable salts of the compounds of this invention are utilized in these compositions, those salts are preferably derived from inorganic or organic acids and bases. Nonlimiting examples of such acid salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate [e.g. (+)-tartrates, (−)-tartrates or mixtures thereof, including racemic mixtures], thiocyanate, tosylate and undecanoate. Base salts include, without limitation, ammonium salts, alkali metal salts, such as sodium and potassium salts, alkaline earth metal salts, such as calcium and magnesium salts, salts with organic bases, such as dicyclohexylamine or N-methyl-D-glucamine, and salts with amino acids such as arginine or lysine.

Also, basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

The disclosed Chk-1 inhibitors are advantageously administered to inhibit Chk-1 in a subject in whom a beneficial therapeutic or prophylactic effect can be achieved by inhibiting Chk-1, i.e., a subject in need of Chk-1 inhibition. A “subject” is a mammal, preferably a human or an animal in need of veterinary treatment, e.g., companion animals (e.g., dogs, cats, and the like), farm animals (e.g., cows, sheep, pigs, horses, and the like), and laboratory animals (e.g., rats, mice, guinea pigs, and the like).

Chk-1 inhibition can be used to achieve a beneficial therapeutic or prophylactic effect, for example, in subjects with cancer. Cancers which can be treated with Chk-1 inhibitors include cancers or cell types (e.g., solid tumors such as colon, breast, lung, ovarian, pancreatic or non-solid tumors such as non-Hodgkins lymphomas and leukemias) in which p53 or the p53 pathway has been inactivated or abrogated. Chk-1 inhibitors are particularly useful in the treatment of cancers or cell types in which Chk-1 protein or activity is up regulated (e.g., retinoblastomas such as Rb negative or inactivated cells (Gottifredi et al., Mol. Cell. Biol., 21:1066 (2001))), or where the p16^{INK4a}/p14^{ARF} locus has been inactivated or misregulated. Use of Chk-1 inhibitors as drugs for the treatment of cancer also is particularly advantageous and can enhance the effectiveness of the treatment when: 1) combined with radiation therapy or chemotherapeutic agents that act by causing damage to the genetic material of cells (collectively referred to herein as “DNA damaging agents”); 2) combined with agents which are otherwise cytotoxic to cancer cells during cell division; 3) combined with agents which are proteasome inhibitors; 4) combined with agents which inhibit NF-κB (e.g., IKK inhibitors) (Bottero et al., Cancer Res., 61:7785 (2001); or 5) used with combinations of cancer drugs with which are not cytotoxic when administered alone, yet in combination produce a toxic effect. Examples of DNA damaging chemotherapeutic agents include topoisomerase I inhibitors (e.g., irinotecan, camptothecin and analogs or metabolites thereof, and doxorubicin); topoisomerase II inhibitors (e.g., etoposide and daunorubicin); alkylating agents (e.g., methotrexate or cyclophosphamide); DNA intercalators (e.g., cisplatin and carboplatin); DNA intercalators and free radical generators such as bleomycin; and nucleoside mimietics (e.g., 5-fluorouracil gemcitabine and hydroxyurea). Agents that disrupt cell replication include: taxol and taxol analogs; vinblastin and vinblastin analogs; antibodies, such as trastuzumab (Herceptin), which bind to proteins overexpressed in cancers and thereby downregulate cell replication; and inhibitors, such as STI-571 (Gleevec), of proteins or enzymes known to be upregulated, over-expressed or activated in cancers, the inhibition of which downregulates cell replication.

The disclosed Chk-1 inhibitors are also effective when used in combination with DNA-damaging anti-cancer drugs and/or radiation therapy to treat subjects with multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, typically five or more.

As such, an “effective amount” of the disclosed Chk-1 inhibitors is the quantity which inhibits Chk-1 when administered to a subject or which, when administered to a subject with cancer, slows tumor growth, ameliorates the symptoms of the disease and/or increases longevity. When used in combination with a DNA damaging agent, an effective amount of the Chk-1 inhibitor is the quantity at which a greater response is achieved when the Chk-1 inhibitor is co-administered with the DNA damaging anti-cancer drug and/or radiation therapy than is achieved when the DNA damaging anti-cancer drug and/or radiation therapy is administered alone. When used as a combination therapy, an “effective amount” of the DNA damaging agent is administered to the subject, which is a quantity that normally produces an anti-cancer effect. The disclosed Chk-1 inhibitors and a DNA damaging chemotherapeutic agent can be co-administered to the subject as part of the same pharmaceutical composition or, alternatively, as separate pharmaceutical compositions. When administered as separately, the Chk-1 inhibitor and the DNA-damaging chemotherapy and/or radiation therapy can be administered simultaneously or at different times, provided that the enhancing effect of the Chk-1 inhibitor is retained.

The amount of Chk-1 inhibitor, DNA damaging agent (chemotherapy and/or radiation therapy) administered to the subject will depend on the type and severity of the disease or condition and on the characteristics of the subject, such as general health, age, sex, body weight and tolerance to drugs. The skilled artisan will be able to determine appropriate dosages depending on these and other factors. Effective dosages for commonly used anti-cancer drugs and radiation therapy are well known to the skilled person. Effective amounts of the disclosed Chk-1 inhibitors typically range between about 1 mg/mm² per day and about 10 grams/mm² per day, and preferably between 10 mg/mm² per day and about 5 grams/mm².

The Chk-1 inhibitors described herein, and the pharmaceutically acceptable salts, solvates and hydrates thereof can be used in pharmaceutical preparations in combination with a pharmaceutically acceptable carrier or diluent. Suitable pharmaceutically acceptable carriers include inert solid fillers or diluents and sterile aqueous or organic solutions. The Chk-1 inhibitor will be present in such pharmaceutical compositions in amounts sufficient to provide the desired dosage amount in the range described herein. Techniques for formulation and administration of the compounds of the instant invention can be found in Remington: the Science and Practice of Pharmacy, 20th edition, ed. A. Gennaro, Lippincott Williams & Wilkins, 2000.

For oral administration, the Chk-1 inhibitor or salts thereof can be combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

For parental administration the disclosed Chk-1 inhibitor, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition, to the formulations described previously, the compounds may also be formulated as a depot preparation. Such long acting formulations may be administered by implantation, for example, subcutaneously or intramuscularly or by intramuscular injection. Thus, for example, as an emulsion in an acceptable oil, or ion exchange resins, or as sparingly soluble derivatives, for example, as sparingly soluble salts.

In some embodiments, a Chk-1 inhibitor of formula (I), or a pharmaceutical formulation containing the Chk-1 inhibitor, is in a unit dosage form for administration to a mammal. The unit dosage form can be any unit dosage form known in the art including, for example, a capsule, an IV bag, a tablet, or a vial. The quantity of active ingredient (viz., a compound of formula (I) or a pharmaceutically acceptable salt thereof) in a unit dose of a pharmaceutical composition is an effective amount, which may be varied according to the particular treatment involved. It will be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

### EXAMPLES

### Example 1

### 2-Amino-N-(3-{4-chloro-2-[3-(5-cyano-pyridin-2-yl)ureido]-phenoxy}-propyl)-3-(4-chloro-phenyl)-propionamide (Compound I-1)

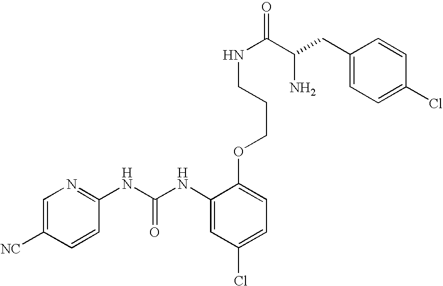 Step 1

### [3-(4-Chloro-2-nitro-phenoxy)-propyl]-carbamic acid tert-butyl ester

A solution of (3-hydroxy-propyl)-carbamic acid tert-butyl ester (11 mmol, 1.88 mL) in THF (20 mL) was added to a suspension of sodium hydride 50% grade (23 mmol, 1.058 g) in THF (20 mL) and stirred at 50° C. for 1 hour. Following cooling to 0° C., 4-chloro-1-fluoro-2-nitro-benzene was added and the mixture stirred at ambient temperature overnight. After quenching with water, the organic layer was separated and the aqueous layer was extracted several times with ethyl acetate. The combined organic layers were washed with water then brine, and dried over sodium sulfate. The crude reaction was purified by flash chromatography to afford [3-(4-chloro-2-nitro-phenoxy)-propyl]-carbamic acid tert-butyl ester.

¹H NMR (400 MHz, DMSO-d₆) δ 8.014 (d, 1H), 7.71 (dd, 1H), 7.37 (d, 1H), 6.85 (t, broad, 1H), 4.16 (t, 2H), 3.06 (td, 2H), 1.85-1.79 (m, 2H), 1.36 (s, 9H). LCMS: method B, retention time (Rₜ)=3.52 min, [MH⁺=331].

Step 2

### [3-(2-Amino-4-chloro-phenoxy)-propyl]-carbamic acid tert-butyl ester

A solution of [3-(4-chloro-2-nitro-phenoxy)-propyl]-carbamic acid tert-butyl ester (1.78 g) was hydrogenated in ethanol (50 mL) in presence of platinum oxide (178 mg) for 2 hours. The mixture was filtrated over a pad of celite and concentrated under vacuum. The crude reaction was purified by flash chromatography to afford [3-(2-amino-4-chloro-phenoxy)-propyl]-carbamic acid tert-butyl ester (1.39 g, 86%) as a viscous oil which solidified slowly:

¹H NMR (400 MHz, DMSO-d₆) δ 6.80 (t, broad, 1H), 6.67 (d, 1H), 6.56 (d, 1H), 6.41 (dd, 1H), 4.92 (s, broad, 2H), 3.86 (t, 2H), 3.03 (td, 2H), 1.79-7.73 (m, 2H), 1.31 (s, 9H). LCMS, method B, Rₜ=3.28 min, [MH⁺=301].

Step 3

### (3-{4-Chloro-2-[3-(5-cyano-pyrdin-2-yl)-ureido]-phenoxy}-propyl)-carbamic acid tert-butyl ester

To a suspension of [3-(2-amino-4-chloro-phenoxy)-propyl]-carbamic acid tert-butyl ester (200 mg, 0.66 mmol) in tetrahydrofuran (2 mL) was added in one portion 1,1′-carbonyldiimidazole (163 mg, 1.01 mmol). The reaction mixture was stirred under nitrogen for 1 hour at ambient temperature. 2-Amino-5-cyanopyridine (256 mg, 2.15 mmol) and tetrahydrofuran (2 mL) were added to the reaction mixture. The resulting mixture was heated in a microwave at 110° C. for 10 minutes (×2). The solvent was removed in vacuo and the resulting solid was triturated with hot methanol. The cooled methanol solution was filtered to afford the title compound as a white solid (55 mg, 18%).

¹H NMR (400 MHz, DMSO-d₆) δ 10.76 (s, 1H, broad), 10.62 (s, 1H, broad), 8.76 (s, 1H), 8.35 (s, 1H), 8.24 (dd, 1H), 7.53 (d, 1H), 7.11 (s, 2H), 6.97 (s, 1H, broad), 4.16 (t, 2H), 3.21 (q, 2H), 2.02 (m, 2H), 1.41 (s, 9H). LCMS: method B, Rₜ=4.15 min, [MNa⁺=468].

Step 4

### 1-[2-(3-Amino-propoxy)-5-chloro-phenyl]-3-(5-cano-pyridin-2-yl)-urea.HCl

To a solution of (3-{4-Chloro-2-[3-(5-cyano-pyrdin-2-yl)-ureido]-phenoxy}-propyl)-carbamic acid tert-butyl ester (55 mg, 0.12 mmol) in methanol (2 mL) was added 4M HCl/dioxane (2 mL). The reaction mixture was stirred for 2 h and the solvent was removed in vacuo. The solid was triturated with dioxane and filtered. The precipitate was washed with 5% methanol in dioxane to yield an off-white solid (44 mg, 93%). LCMS: method B, Rₜ=3.41 min, [MH⁺=346].

Step 5

### [1-(3-{4-Chloro-2-[3-(5-cyano-pyridin-2-yl)-ureido]-phenoxy}-propylcarbamoyl)-2-(4-chloro-phenyl)-ethyl]-carbamic acid tert-butyl ester

To a stirred solution of 1-[2-(3-amino-propoxy)-5-chloro-phenyl]-3-(5-cyano-pyridin-2-yl)-urea•HCl (44 mg, 0.12 mmol) and diisopropylethylamine (47.7 mg, 0.369 mmol) in dimethylformamide (2 mL) was added Boc-Phe-(4-Cl)—OH (69 mg, 0.231 mmol) and 1-hydroxybenzotriazole (39 mg, 0.288 mmol). 1-(3-Dimethylaminopropyl)-3-ethylcarbodiimide hydrochloride (44 mg, 0.231 mmol) was added to the reaction mixture and stirred overnight. Addition of water resulted in the formation of a precipitate which was collected by filtration. The solid was partitioned between ethyl acetate and water. The organic layer was collected, dried (MgSO₄) and filtered. The solvent was removed in vacuo to yield a white solid (56 mg, 78%).

Step 6

### 2-Amino-N-(3-{4-chloro-2-[3-(5-cyano-pyridin-2-yl)ureido]-phenoxy}-propyl)-3-(4-chloro-phenyl)-propionamide

To a solution of [1-(3-{4-chloro-2-[3-(5-cyano-pyridin-2-yl)-ureido]-phenoxy}-propylcarbamoyl)-2-(4-chloro-phenyl)-ethyl]-carbamic acid tert-butyl ester (44 mg, 0.077 mmol) in methanol (2 mL) was added 4M HCl/dioxane (2 mL). The reaction mixture was stirred for 2 hours and the solvent was removed in vacuo. The solid was triturated with dioxane and filtered. The precipitate was washed with 5% methanol in dioxane to yield a white solid (17 mg, 43%). LCMS: method B, Rₜ=4.20 min, [MH⁺=527].

### Example 2

### 6-[3-(2-{3-[2-Amino-3-(4-chloro-phenyl)-propionylamino]-propoxy}-5-chloro-phenyl)-ureido]-nicotinamide (Compound I-2)

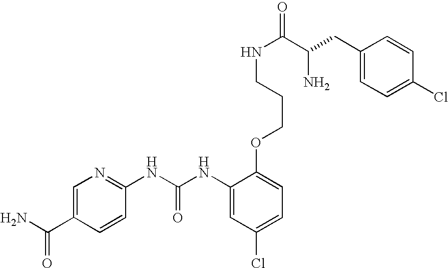

The title compound was prepared from the appropriate reagents, using a procedure similar to that described in Example 1. ¹H NMR (400 MHz, MeOD) δ 8.65 (s, 1H, broad), 8.21 (d, 1H), 8.15 (d, 1H), 7.21 (d, 2H), 7.14 (m, 3H), 6.92 (dd, 1H), 6.85 (d, 1H), 4.10-3.80 (m, 3H), 3.45-3.28 (m, 2H), 3.10-2.95 (m, 2H), 1.96 (m, 2H). LCMS: method B, Rₜ=3.99 min, [MH⁺=545].

### Example 3

### N-(3-{4-Chloro-2-[3-(5-methyl-pyrazin-2-yl)-ureido]-phenoxy}-propyl)-4-dimethylamino-benzamide (Compound I-3)

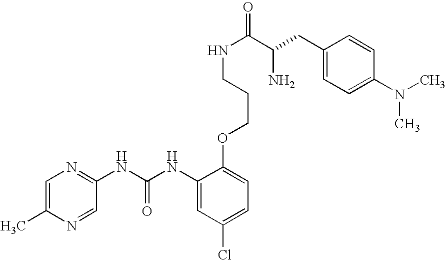

To a stirred solution of 1-[2-(3-amino-propoxy)-5-chloro-phenyl]-3-(5-methyl-pyrazin-2-yl)-urea (20 mg, 0.054 mmol), prepared by procedures analogous to those described in Example 1, and diisopropylethylamine (13.5 mg, 0.108 mmol) in dichloromethane (1 mL) was added (4-dimethylamino)benzoyl chloride (10 mg, 0.054 mmol). The reaction mixture was stirred for 48 hours. The resulting precipitate was collected by filtration and dried under vacuum. The solid was re-dissolved in 4M HCl/dioxane (0.5 mL) and methanol (0.5 mL) and allowed to stand overnight to yield the title compound as a yellow crystalline solid after filtration (16.6 mg, 58%).

¹H NMR (400 MHz, DMSO-d₆) δ 10.27 (s, 1H), 8.68 (s, 1H), 8.31 (d, 2H), 8.20 (s, 1H), 7.77 (d, 2H), 7.03 (m, 2H), 6.80 (d, 2H), 4.14 (t, 2H), 3.48 (m, 2H), 2.97 (s, 6H), 2.40 (s, 3H), 2.11 (m, 2H). LCMS: method B, Rₜ=4.01 min, [MH⁺=483].

### Example 4

### N-(3-{4-Chloro-2-[3-(5-methyl-pyrazin-2-yl)-ureido]-phenoxy}-propyl)-2-dimethylamino-acetamide (Compound I-4)

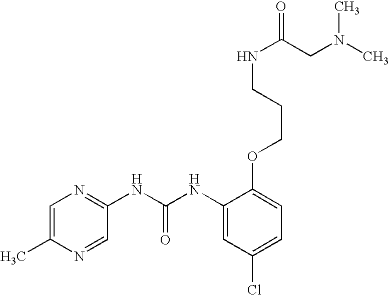

To a stirred solution of 1-[2-(3-amino-propoxy)-5-chloro-phenyl]-3-(5-methyl-pyrazin-2-yl)-urea (20 mg, 0.054 mmol) and diisopropylethylamine (27 mg, 0.216 mmol) in dichloromethane (1 mL) was added chloroacetyl chloride (10 mg, 0.054 mmol). The reaction mixture was stirred for 1 hour before adding dimethylamine (144 mg) and diisopropylethylamine (0.5 mL). The reaction mixture was heated in a microwave at 100° C. for 10 minutes. The solvent was removed in vacuo and the resulting solid partitioned between ethyl acetate and water. The organic layer was collected, dried (MgSO₄) and filtered. The solvent was removed in vacuo to yield a solid which was triturated with ethyl acetate and hexane (80:20) to afford the title compound as an off-white solid (9 mg, 41%).

¹H NMR (400 MHz, MeOD) δ 8.45 (s, 1H), 8.18 (s, 1H), 8.07 (s, 1H), 6.88 (s, 2H), 4.04 (t, 2H), 3.42 (t, 2H), 3.21 (s, 6H), 2.39 (s, 2H), 2.38 (s, 3H), 2.04 (m, 2H).

### Example 5

### Morpholine-4-carboxylic acid (3-{4-chloro-2-[3-(5-methyl-pyrazin-2-yl)-ureido]-phenoxy}-propyl)-amide (Compound I-5)

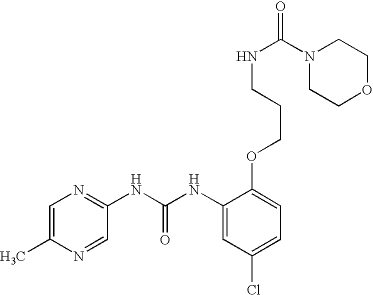

The title compound was prepared from the appropriate reagents, using procedures similar to those described in Example 12. LCMS: method B, Rₜ=4.01 min, [MH⁺=483].

### Example 6

### N-(3-{4-Chloro-2-[3-(5-methyl-pyrazin-2-yl)-ureido]-phenoxy}-propyl)-2-morpholin-4-yl-acetamide (Compound I-6)

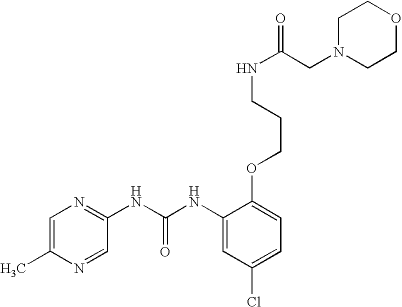

The title compound was prepared from the appropriate reagents, using a procedure similar to that described in Example 4. ¹H NMR (400 MHz, MeOD) δ 8.58 (s, 1H), 8.31 (s, 1H), 8.19 (s, 1H), 6.99 (s, 2H), 4.15 (t, 2H), 3.66 (t, 4H), 3.50 (t, 2H), 3.01 (s, 2H), 2.50 (s, 3H), 2.49 (m, 4H), 2.15 (m, 2H).

### Example 7

### N-(3-{4-Chloro-2-[3-(5-methyl-pyrazin-2-yl)-ureido]-phenoxy}-propyl)-2-(4-methyl-piperazin-1-yl)-acetamide (Compound I-7)

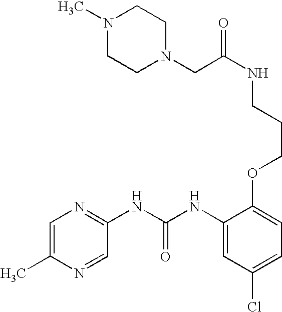

The title compound was prepared from the appropriate reagents, using a procedure similar to that described in Example 4. ¹H NMR (400 MHz, MeOD) δ 8.44 (s, 1H), 8.19 (s, 1H), 8.04 (s, 1H), 6.87 (d, 2H), 4.03 (t, 2H), 3.42 (t, 2H), 2.89 (s, 2H), 2.50-2.22 (m, 8H), 2.48 (s, 3H), 2.13 (s, 3H), 2.03 (m, 2H).

### Example 8

### 1-[5-Chloro-2-(2-morpholin-4-yl-2-oxo-ethoxy)-phenyl]-3-(5-methyl-pyrazin-2-yl)-urea (Compound I-8)

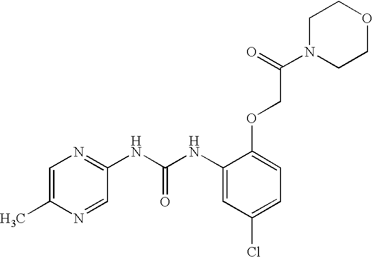 Step 1

### 2-(4-Chloro-2-nitro-phenoxy)-1-morpholin-4-yl-ethanone

To a suspension of 4-chloro-2-nitro-phenol (500 mg, 2.90 mmol) and potassium carbonate (1.60 g, 11.6 mmol) in DMF (20 mL) was added in one portion 2-chloro-1-morpholin-4-yl-ethanone (709 mg, 4.30 mmol). The reaction mixture was heated at 70° C. overnight under a nitrogen atmosphere. The reaction mixture was diluted with ethyl acetate and washed with water several times. The organic layer was then dried (MgSO₄) and concentrated to give a yellow solid (790 mg, 91%).

¹H NMR (400 MHz, DMSO-d₆) δ 8.03 (d, 1H), 7.70 (dd, 1H), 7.31 (d, 1H), 5.15 (s, 2H), 3.62 (m, 2H), 3.58 (m, 2H), 3.43 (m, 4H). LCMS: method A, Rₜ=2.70 min, [MH⁺=301].

Step 2

### 2-(2-Amino-4-chloro-phenoxy)-1-morpholin-4-yl-ethanone

The title compound was prepared from 2-(4-(chloro-2-nitro-phenoxy)-1-morpholin-4-yl-ethanone by a procedure analogous to that described in Example 1, step 2. ¹H NMR (400 MHz, DMSO-d₆) δ 6.75 (d, 1H), 6.67 (s, 1H), 6.49 (d, 1H), 5.11 (s, 2H, broad), 4.79 (s, 2H), 3.59 (m, 4H), 3.46 (m, 4H). LCMS: method A, Rₜ=2.26 min, [MH⁺=271].

Step 3

### 1-[5-Chloro-2-(2-morpholin-4-yl-2-oxo-ethoxy)-phenyl]-3-(5-methyl-pyrazin-2-yl)-urea

The title compound was prepared from the appropriate reagents, using a procedure similar to that described in Example 1, step 3. ¹H NMR (400 MHz, DMSO-d₆) δ 10.39 (s, 1H, broad), 10.17 (s, 1H), 8.74 (s, 1H), 8.32 (s, 1H), 8.25 (s, 1H), 7.03 (d, 1H), 7.02 (d, 1H), 5.04 (s, 2H), 3.60 (m, 4H), 3.49 (m, 4H), 2.45 (s, 3H). LCMS: method A, Rₜ=2.68 min, [MH⁺=406].

### Example 9

### 4-[3-(4-Chloro-benzoylamino)-propoxy]-N-(2-dimethylamino-ethyl)-3-[3-(5-methyl-pyrazin-2-yl)-ureido]-benzamide hydrochloride salt (Compound I-9)

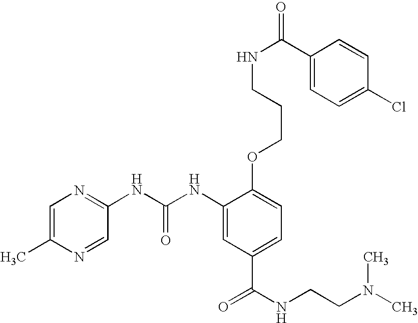 Step 1

### 4-(3-tert-Butoxycarbonylamino-propoxy)-3-nitro-benzoic acid ethyl ester

Ethyl 4-hydroxy-3-nitrobenzoate (5.0 g, 24 mmol), N-Boc-3-bromopropylamine (11.4 g, 48.8 mmol), and potassium carbonate (13 g, 95 mmol) were added to DMF and stirred at 70° C. overnight. The reaction mixture was then diluted with ethyl acetate and washed with water. The organic layer was dried (MgSO₄), concentrated in vacuo, and recrystallized from ethyl acetate to give a white solid (7.9 g, 90%).

¹H NMR (400 MHz, CDCl₃) δ 8.46 (d, 1H), 8.14 (dd, 1H), 7.05 (d, 1H), 4.90 (s, 1H, broad), 4.32 (q, 2H), 4.17 (t, 2H), 3.30 (m, 2H), 2.00 (m, 2H), 1.40 (s, 9H), 1.33 (3H, t). LCMS: method A, Rₜ=3.45 min, [M+Na⁺=391].

Step 2

### 4-(3-Amino-propoxy)-3-nitro-benzoic acid ethyl ester hydrochloride salt

4-(3-tert-Butoxycarbonylamino-propoxy)-3-nitro-benzoic acid ethyl ester (1.5 g, 4.1 mmol) was dissolved in 4N HCl in dioxane (100 mL) and stirred for 2 h. The solution was concentrated in vacuo to give a yellow solid (1.19 g, 92%). ¹H NMR (400 MHz, CDCl₃) δ 8.46 (s, 1H), 8.18 (s, 2H, broad), 8.14 (d, 1H), 7.05 (d, 1H), 4.30 (q, 2H), 3.80 (t, 2H), 3.72 (m, 2H), 1.95 (m, 2H), 1.33 (3H, t). LCMS: method A, Rₜ=1.75 min, [MH⁺=269].

Step 3

### 4-[3-(4-Chloro-benzoylamino)-propoxy]-3-nitro-benzoic acid ethyl ester

4-(3-Amino-propoxy)-3-nitro-benzoic acid ethyl ester hydrochloride salt (1.19 g, 3.9 mmol), 4-chloro-benzoyl chloride (0.73 mL, 5.8 mmol), and diisopropylethylamine (2 mL, 2.5 mmol) were dissolved in dichloromethane (30 mL) and stirred overnight. The reaction mixture was diluted with dichloromethane, washed with water, dried (MgSO₄), and concentrated in vacuo to afford a yellow solid. The residue was purified by column chromatography (40% ethyl acetate/hexane) to afford a white solid (1.23 g, 77%).

¹H NMR (400 MHz, CDCl₃) δ 8.51 (d, 1H), 8.22 (dd, 1H), 7.73 (d, 2H), 7.39 (d, 2H), 7.13 (d, 1H), 6.74 (s, 1H, broad), 4.39 (q, 2H), 4.32 (t, 2H), 3.72 (q, 2H), 2.23 (m, 2H), 1.40 (t, 3H). LCMS: method A, Rₜ=3.45 min, [MH⁺=407].

Step 4

### 3-Amino-4-[3-(4-chloro-benzoylamino)-propoxy]-benzoic acid ethyl ester

4-[3-(4-Chloro-benzoylamino)-propoxy]-3-nitro-benzoic acid ethyl ester (1.23 g, 3.26 mmol) was dissolved in ethanol (100 mL) and ethyl acetate (20 mL) in the presence of 10% wt. platinum on carbon (120 mg) and stirred under a hydrogen atmosphere for 4 hours. The mixture was then filtered through Celite and concentrated in vacuo to afford a white solid (1.15 g, 94%). LCMS: method A, Rₜ=3.00 min, [MH⁺=378].

Step 5

### 4-[3-(4-Chloro-benzoylamino)-propoxy]-3-[3-(5-methyl-pyrazin-2-yl)-ureido]-benzoic acid ethyl ester

5-Methyl-pyrazine-2-carboxylic acid (0.5 g, 3 mmol) was heated in toluene (50 mL) with stirring. Diphenyl phosphoryl azide (DPPA) was added. The temperature was increased to 100° C. over 10 minutes and maintained for a further 15 minutes, until the evolution of gas had ceased. During this time, the solution changed colour from orange to dark red/brown. 3-Amino-4-[3-(4-chloro-benzoylamino)-propoxy]-benzoic acid ethyl ester (1.15 g, 3 mmol), in toluene (20 mL), was added via syringe at 100° C. and stirred for 30 minutes. The heat was removed and the reaction mixture was cooled to room temperature. The resulting precipitate was collected via filtration to give a white solid (888 mg, 58%).

¹H NMR (400 MHz, DMSO-d₆) δ 10.17 (s, 1H), 8.87 (s, 1H), 8.65 (s, 1H), 8.66 (s, 1H, broad), 8.17 (s, 1H), 7.86 (d, 2H), 7.65 (dd, 1H), 7.52 (d, 2H), 7.15 (d, 1H), 4.30 (q, 2H), 4.24 (t, 2H), 3.52 (m, 2H), 2.39 (s, 3H), 2.16 (m, 2H), 1.31 (t, 3H). LCMS: method A, Rₜ=3.33 min, [MH⁺=512].

Step 6

### 4-[3-(4-Chloro-benzoylamino)-propoxy]-3-[3-(5-methyl-pyrazin-2-yl)-ureido]-benzoic acid

4-[3-(4-Chloro-benzoylamino)-propoxy]-3-[3-(5-methyl-pyrazin-2-yl)-ureido]-benzoic acid ethyl ester (88 mg, 1.7 mmol) and sodium hydroxide were dissolved in methanol (30 mL), water (45 mL), and tetrahydrofuran (15 mL) and stirred at 65° C. overnight. The volatile solvents were removed in vacuo and the aqueous residue was washed with ethyl acetate and then acidified to pH 6 using 1N HCl. The resulting white precipitate was collected by filtration (747 mg, 91%).

¹H NMR (400 MHz, DMSO-d₆) δ 10.25 (s, 1H), 10.12 (s, 1H, broad), 8.74 (t, 1H), 8.69 (s, 1H), 8.36 (s, 1H), 8.19 (s, 1H), 7.89 (d, 2H), 7.51 (d, 2H), 7.11 (m, 2H), 4.20 (t, 2H), 2.77 (m, 2H), 2.39 (s, 3H), 2.15 (m, 2H). LCMS: method A, Rₜ=2.76 min, [MH⁺=484].

Step 7

### 4-[3-(4-Chloro-benzoylamino)-propoxy]-N-(2-dimethylamino-ethyl)-3-[3-(5-methyl-pyrazin-2-yl)-ureido]-benzamide hydrochloride salt

4-[3-(4-Chloro-benzoylamino)-propoxy]-3-[3-(5-methyl-pyrazin-2-yl)-ureido]-benzoic acid (100 mg, 0.2 mmol), hydroxybenzotriazole (81 mg, 0.6 mmol), and diisopropylethylamine (0.1 mL, 0.6 mmol) were dissolved in DMF (5 mL) under a nitrogen atmosphere. N,N-Dimethylethylenediamine (0.064 mL, 0.60 mmol) and 1-(3-dimethylaminopropyl)-3-ethylcarbodiimide hydrochloride (115 mg, 0.6 mmol) were then added and stirred at 70° C. for 2 hours. The reaction mixture was diluted with ethyl acetate and washed with water. A solid precipitated in the organic layer, which was concentrated in vacuo. The resulting residue was stirred in 1.25 M HCl in methanol (10 mL), concentrated in vacuo, and triturated with ethyl acetate to afford a white solid (77 mg, 75%).

¹H NMR (400 MHz, DMSO-d₆) δ 10.45 (s, 1H, broad), 10.25 (s, 1H), 8.79 (t, 1H), 8.73 (m, 1H), 8.69 (m, 1H), 8.18 (s, 1H), 7.90 (d, 2H), 7.64 (dd, 1H), 7.50 (d, 2H), 7.10 (d, 1H), 4.22 (t, 2H), 3.63 (m, 2H), 3.52 (m, 2H), 3.25 (m, 2H), 2.82 (s, 3H), 2.81 (s, 3H), 2.39 (s, 3H), 2.15 (m, 2H). LCMS: method A, Rₜ=1.95 min, [MH⁺=554].

### Example 10

### 4-Chloro-N-(3-{2-[3-(5-methyl-pyrazin-2-yl)-ureido]-5-pyrrolidin-1-yl-phenoxy}-propyl)-benzamide hydrochloride salt (Compound I-10)

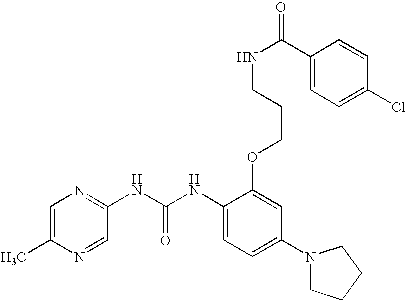 Step 1

### [3-(5-Fluoro-2-nitro-phenoxy)-propyl]-carbamic acid tert-butyl ester

The title compound was prepared from the appropriate reagents, using a procedure similar to that described in Example 9, step 1. ¹H NMR (400 MHz, DMSO-d₆) δ 8.00 (dd, 1H), 6.79 (dd, 1H), 6.74 (td, 1H), 5.04 (s, 1H, broad), 4.18 (t, 2H), 3.39 (m, 2H), 2.09 (t, 2H), 1.45 (s, 9H). LCMS: method A, Rₜ=3.27 min, [MH⁺=315, MNa⁺=337].

Step 2

### [3-(2-Nitro-5-pyrrolidin-1-yl-phenoxy)-propyl]-carbamic acid tert-butyl ester

To a solution of [3-(5-fluoro-2-nitro-phenoxy)-propyl]-carbamic acid tert-butyl ester (1.0 g, 3.2 mmol) and pyrrolidine (0.40 mL, 4.8 mmol) in THF (10 mL) was added potassium carbonate (658 mg, 4.78 mmol). The mixture was refluxed for 6 hours. After cooling, water was added and the product was extracted with ethyl acetate (3×). The organic extracts were dried (MgSO₄) and concentrated to a yellow solid (1.14 g, 98%). LCMS: method A, Rₜ=3.48 min, [MH⁺=366].

Step 3

### [3-(2-Amino-5-pyrrolidin-1-yl-phenoxy)-propyl]-carbamic acid tert-butyl ester

[3-(2-Nitro-5-pyrrolidin-1-yl-phenoxy)-propyl]-carbamic acid tert-butyl ester (1.72 g, 4.71 mmol) and 10% wt. palladium on carbon catalyst (180 mg) were stirred in methanol under an atmosphere of hydrogen for 4 hours and then filtered through a pad of Celite. The filtrate was concentrated to yield a dark oil (1.63 g, 100%).

¹H NMR (400 MHz, DMSO-d₆) δ 6.69 (d, 1H), 6.17 (s, 1H), 6.08 (d, 1H), 4.82 (s, 1H, broad), 3.98 (t, 2H), 3.27 (m, 2H), 3.22 (m, 4H), 1.99 (m, 6H), 1.45 (s, 9H). LCMS: method A, Rₜ=1.96 min, [MH⁺=336].

Step 4

### [3-{2-[3-(5-Methyl-pyrazin-2-yl)-ureido]-5-pyrrolidin-1-yl-phenoxy}-propyl)-carbamic acid tert-butyl ester

The title compound was prepared from the appropriate reagents, using a procedure similar to that described in Example 1, step 3. ¹H NMR (400 MHz, DMSO-d₆) δ 9.83 (s, 2H, broad), 8.63 (s, 1H), 8.13 (s, 1H), 7.90 (d, 1H), 6.92 (s, 1H, broad), 6.21 (s, 1H), 6.09 (d, 1H), 4.07 (t, 2H), 3.17-3.25 (m, 6H), 2.40 (s, 3H), 1.96 (m, 6H), 1.38 (s, 9H). LCMS: method A, Rₜ=3.22 min, [MH⁺=471].

Step 5

### 4-Chloro-N-(3-{2-[3-(5-methyl-pyrazin-2-yl)-ureido]-5-pyrrolidin-1-yl-phenoxy}-propyl)-benzamide hydrochloride salt

[3-{2-[3-(5-Methyl-pyrazin-2-yl)-ureido]-5-pyrrolidin-1-yl-phenoxy}-propyl)-carbamic acid tert-butyl ester (200 mg, 0.43 mmol) was stirred in 4M HCl-dioxane (5 mL) for 1 hour. The reaction mixture was concentrated to a brown solid and then dissolved in DCM (5 mL) and diisopropylethylamine (0.37 mL, 2.15 mmol) before the addition of 4-chlorobenzoyl chloride (0.082 mL, 0.65 mmol). After 2 hours the reaction was filtered and washed with DCM to yield a yellow solid (152 mg, 70%). The product was treated with 1.25 M HCl in methanol (3 mL) and stirred for 15 minutes. The resultant solid was collected by filtration and washed with methanol to yield a grey solid (138 mg, 64%).

¹H NMR (400 MHz, DMSO-d₆) 69.82 (s, 2H, broad), 8.62 (s, 1H), 8.62 (s, 1H, broad), 8.13 (s, 1H), 7.88 (d, 1H), 7.87 (d, 2H), 7.52 (d, 2H), 6.21 (s, 1H), 6.08 (d, 1H), 4.12 (t, 2H), 3.49 (m, 2H), 3.18 (m, 4H), 2.36 (s, 3H), 2.10 (m, 2H), 1.92 (m, 4H). LCMS: method A, Rₜ=3.09 min, [MH⁺=509].

### Example 11

### 2-Dimethylamino-N-(3-{2-[3-(5-methyl-pyrazin-2-yl)-ureido]-5-pyrrolidin-1-yl-phenoxy}-propyl)-acetamide hydrochloride salt (Compound I-11)

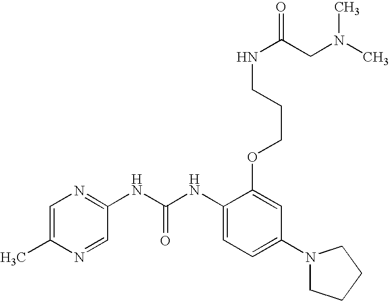

[3-{2-[3-(5-Methyl-pyrazin-2-yl)-ureido]-5-pyrrolidin-1-yl-phenoxy}-propyl)-carbamic acid tert-butyl ester (from Example 10, step 4) was deprotected and converted to the title compound using the appropriate reagents, using a procedure similar to that described in Example 10, step 5.

¹H NMR (400 MHz, DMSO-d₆) δ 10.12 (s, 1H, broad), 9.89 (s, 1H, broad), 8.84 (t, 1H, broad), 8.71 (s, 1H), 8.17 (s, 1H), 8.05 (d, 1H), 6.85 (s, 1H, broad), 6.70 (d, 1H, broad), 4.22 (t, 2H), 3.92 (m, 2H), 3.44 (m, 6H), 2.79 (s, 3H), 2.78 (s, 3H), 2.44 (s, 3H), 2.05 (m, 6H). LCMS: method B, Rₜ=3.85 min, [MH⁺=456].

### Example 12

### Morpholine-4-carboxylic acid (3-{4-chloro-2-[3-(5-cyano-pyridin-2-yl)-ureido]-phenoxy}-propyl)-amide (Compound I-12)

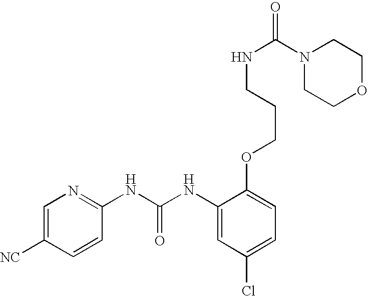 Step 1

### (5-Cyano-pyridin-2-yl)-carbamic acid phenyl ester

Phenylchloroformate (10.5 mL, 83.9 mmol) was added dropwise to a solution of 2-amino-5-cyanopyridine (10.0 g, 83.9 mmol) in THF (200 mL) and pyridine (8.47 mL, 105 mmol) at 0° C. The reaction was stirred under an atmosphere of nitrogen for 30 minutes. The precipitate was filtered, washed with water several times followed by diethyl ether, and then air dried to yield a white solid (19.5 g, 97%).

¹H NMR (400 MHz, DMSO-d₆) δ 8.79 (s, 1H), 8.25 (d, 1H), 7.94 (d, 1H), 7.45 (t, 2H), 7.24-7.26 (m, 3H). LCMS: method A, Rₜ=2.94 min, [MH⁺=240].

Step 2

### (3-{4-Chloro-2-[3-(5-cyano-pyridin-2-yl)-ureido]-phenoxy}-propyl)-carbamic acid tert-butyl ester

Diisopropylethylamine (1.46 mL, 8.37 mmol) was added to a stirred solution of (5-cyano-pyridin-2-yl)-carbamic acid phenyl ester (2.00 g, 8.37 mmol) and [3-(2-amino-4-chloro-phenoxy)-propyl]-carbamic acid tert-butyl ester (2.51 g, 8.37 mmol) in DMSO (40 mL) under an atmosphere of nitrogen. The reaction mixture was stirred at room temperature overnight, during this time a precipitate appeared. Methanol was added to the reaction mixture to form a thick white precipitate which was collected by filtration to yield a white solid (3.0 g, 80%).

¹H NMR (400 MHz, DMSO-d₆) δ 10.70 (s, 1H, broad), 10.55 (s, 1H, broad), 8.70 (s, 1H), 8.29 (s, 1H), 8.17 (d, 1H), 7.45 (d, 1H), 7.03 (s, 2H, broad), 6.90 (s, 1H, broad), 4.11 (t, 2H), 3.15 (m, 2H), 1.97 (m, 2H), 1.35 (s, 9H). LCMS: method A, Rₜ=3.56 min, [MH⁺=446].

Step 3

### Morpholine-4-carboxylic acid (3-{4-chloro-2-[3-(5-cyano-pyridin-2-yl)-ureido]-phenoxy}-propyl)-amide

(3-{4-Chloro-2-[3-(5-cyano-pyridin-2-yl)-ureido]-phenoxy}-propyl)-carbamic acid tert-butyl ester was deprotected and converted to the title compound using the appropriate reagents, using a procedure similar to that described in Example 10, step 5.

¹H NMR (400 MHz, DMSO-d₆) δ 10.70 (s, 1H, broad), 10.53 (s, 1H, broad), 8.70 (s, 1H), 8.28 (s, 1H), 8.16 (d, 1H), 7.47 (d, 1H), 7.03 (s, 2H, broad), 6.60 (t, 1H, broad), 4.08 (t, 2H), 3.51 (t, 4H), 3.20-3.40 (m, 6H), 1.96 (m, 2H). LCMS: method A, Rₜ=3.10 min, [MH⁺=459].

### Example 13

### 4-Chloro-N-(3-{5-(4-methyl-piperazin-1-yl)-2-[3-(5-methyl-pyrazin-2-yl)-ureido]-phenoxy}-propyl)-benzamide (Compound I-13)

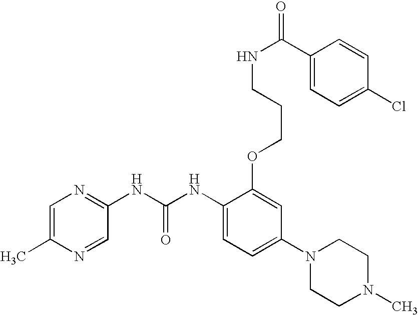

The title compound was prepared from the appropriate reagents, using procedures similar to those described in Example 10. ¹H NMR (400 MHz, DMSO-d₆) δ 9.68 (s, 1H), 8.38 (s, 2H, broad), 7.89 (s, 1H), 7.73 (d, 1H), 7.63 (d, 2H), 6.38 (s, 1H), 6.22 (d, 1H), 3.88 (m, 2H), 3.26 (m, 2H), 2.82 (m, 4H), 2.18 (m, 4H), 2.12 (s, 3H), 1.96 (s, 3H), 1.84 (m, 2H). LCMS: method A, Rₜ=1.90 min, [MH⁺=538].

### Example 14

### 1-{5-Chloro-2-[4-(4-methyl-piperazin-1-yl)-4-oxo-butoxy]-phenyl}-3-(5-cyano-pyridin-2-yl)-urea (Compound I-14)

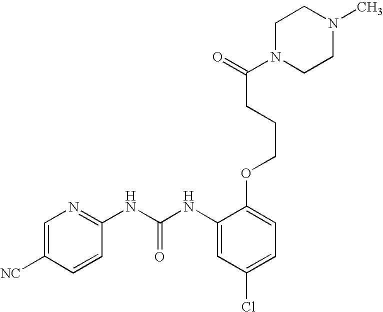 Step 1

### 4-{4-Chloro-2-[3-(5-cyano-pyridin-2-yl)-ureido]-phenoxy}-butyric acid ethyl ester

The title compound was prepared from the appropriate reagents, procedures similar to those described in Example 1 steps 1, 2, and 3. ¹H NMR (400 MHz, DMSO-d₆) δ 10.65 (s, 1H, broad), 10.56 (s, 1H), 8.65 (m, 1H), 8.25 (m, 1H), 8.17 (dd, 1H), 7.46 (d, 1H), 7.03 (m, 2H), 4.10 (dd, 2H), 4.04 (q, 2H), 2.51 (t, 2H), 2.06 (m, 2H), 1.13 (t, 3H).

LCMS: Method A, Rₜ=4.16 min, [MH⁺=403, MNa⁺=425]

Step 2

### 4-{4-Chloro-2-[3-(5-cyano-pyridin-2-yl)-ureido]-phenoxy}-butyric acid

4-{4-Chloro-2-[3-(5-cyano-pyridin-2-yl)-ureido]-phenoxy}-butyric acid ethyl ester (38.5 mg, 0.095 mmol) in a mixture of tetrahydrofuran: (1M KOH/methanol):water (17:6:1) was stirred at room temperature overnight. The reaction mixture was concentrated under vacuum and acidified with 1M HCl. The solution was filtered to afford a white solid (28 mg, 78%).

¹H NMR (400 MHz, DMSO-d₆) δ 10.70 (s, 1H, broad), 10.60 (s, 1H), 8.71 (d, 1H), 8.29 (d, 1H), 8.20 (dd, 1H), 7.50 (d, 1H), 7.00-7.10 (m, 2H), 4.11 (dd, 2H), 2.46 (t, 2H), 2.02-2.12 (m, 2H). LCMS: Method A, Rₜ=3.81 min.

Step 3

### 1-{5-Chloro-2-[4-(4-methyl-piperazin-1-yl)-4-oxo-butoxy]-phenyl}-3-(5-cyano-pyridin-2-yl)-urea

To a suspension of 4-{4-chloro-2-[3-(5-cyano-pyridin-2-yl)-ureido]-phenoxy}-butyric acid (23.5 mg, 0.063 mmol) in a mixture of dichloromethane:dimethylformamide (1:1) was added 1-hydroxybenzotriazole (10.4 mg, 0.076 mmol) and N-methylpiperazine (8 μl, 0.076 mmol. The reaction mixture was stirred for 5 minutes before adding ethyl-(N′,N′-dimethyl amino)propylcarbodiimide hydrochloride (14.7 mg, 0.076 mmol). The reaction mixture was stirred overnight. Water was added and the desired urea precipitated from the reaction mixture. The solution was filtered to afford a white solid (9.2 mg, 32%).

LCMS: Rₜ=4.00 min., [MH⁺=457, MNa⁺=479]

### Example 15

### N-(3-{4-Chloro-2-[3-(5-cyano-pyridin-2-yl)-ureido]-phenoxy-propyl)-2-dimethylamino-acetamide (Compound I-17)

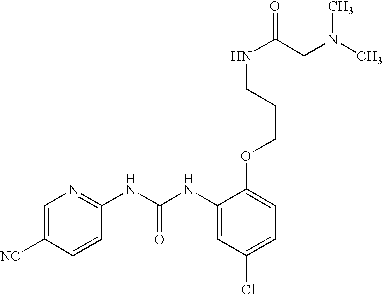

1-[2-(3-Amino-propoxy)-5-chloro-phenyl]-3-(5-cyano-pyridin-2-yl)-urea (200 mg, 0.577 mmol) was suspended in THF (5 mL) over potassium carbonate (175 mg, 1.27 mmol). Dimethylaminoacetyl chloride (96 mg, 0.606 mmol) was added and the reaction was stirred at room temperature for 2 hours. At this time, LCMS indicated complete consumption of starting material to give the desired amide. The potassium carbonate was removed by filtration and the solvent removed in vacuo. The resulting tan solid was triturated in ethyl acetate and the solvent was removed by filtration to give the title compound as a white solid (111 mg, 45%). LCMS: Method B, Rₜ=1.51 min, [MH⁺=431.2].

### Example 16

### 4-Methyl-piperazine-1-carboxylic acid (3-{4-chloro-2-[3-(5-cyano-pyridin-2-yl)-ureido]-phenoxy-propyl)-amide (Compound I-18)

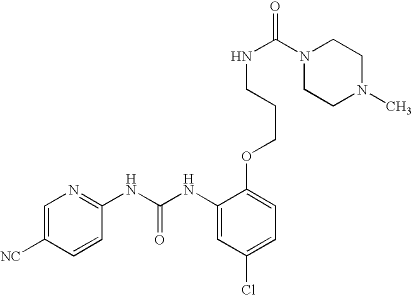

N-Methyl piperidine (70 μL, 0.635 mmol) was slowly added to a pre-stirred solution of triphosgene (280 mg, 0.953 mmol) in methylene chloride (5 mL) over sodium bicarbonate (267 mg, 3.18 mmol) at 0° C. This mixture was stirred for 1.5 h at room temperature until gas evolution had ceased, at which time solid potassium carbonate (239 mg, 1.731 mmol) was added, followed by 1-[2-(3-amino-propoxy)-5-chloro-phenyl]-3-(5-cyano-pyridin-2-yl)-urea (200 mg, 0.577 mmol) in methylene chloride (1 mL). The mixture was allowed to stir at room temperature for 3 days, at which point LCMS indicated disappearance of starting material and conversion to the desired product. The solids were removed by filtration, and the solvent was removed in vacuo. The resulting tan solid was triturated in ethyl acetate and the solvent was removed by filtration to give the title compound as a white solid (112 mg, 41%).

LCMS: Method B, Rₜ=1.49 min, [MH⁺=472.3].

### Example 17

### 1-(5-Chloro-2-{3-[3-(4-dimethylamino-benzyl)-ureido]-propoxy-phenyl)-3-(5-cyano-pyridin-2-yl)-urea (Compound I-96)

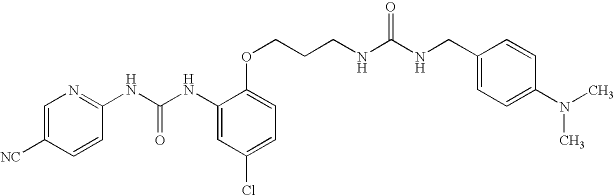

The title compound was prepared from the appropriate reagents, using a procedure similar to that described in Example 16. LCMS: Method A, Rₜ=1.52 nin, [MH⁺=522.2].

### Example 18

### Pyrrolidine-1-carboxylic acid (3{-4-chloro-2-[3-(5-cyano-pyridin-2-yl)-ureido]-phenoxy-propyl)-amide (Compound I-78)

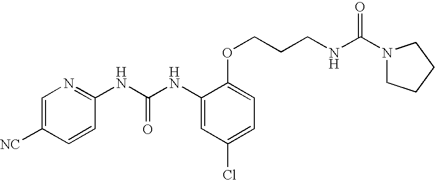

The title compound was prepared from the appropriate reagents, using a procedure similar to that described in Example 16. LCMS: Method A, Rₜ=1.91 min, [MH⁺=443.2].

### Example 19

### (3-{4-Chloro-2-[3-(5-cyano-pyridin-2-yl)-ureido]-phenoxy}-propyl)-carbamic acid 2-methoxy-ethyl ester (Compound I-97)

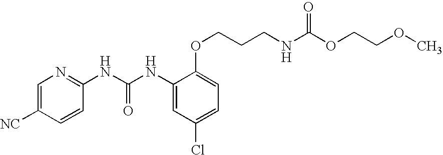

1-[2-(3-Amino-propoxy)-5-chloro-phenyl]-3-(5-cyano-pyridin-2-yl)-urea (50 mg, 0.131 mmol) was suspended in THF (2 mL) over potassium carbonate (44 mg, 0.320 mmol). Chloroformic acid 2-methoxyethyl ester (20 μL, 0.144) was added and the reaction was stirred at room temperature for 2 hours. At this time, LCMS indicated complete consumption of starting material to give the desired carbamide. The potassium carbonate was removed by filtration and the solvent removed in vacuo. The resulting tan solid was triturated in ethyl acetate and the solvent was removed by filtration to give the title compound as a white solid. LCMS: Method A, Rₜ=1.83 min, [MH⁺=448.2].

### Example 20

### 1-(5-Chloro-2-{3-[3-(2-chloro-ethyl)-ureido]-propoxy}-phenyl)-3-(5-cyano-pyridin-2-yl)-urea (Compound I-98)

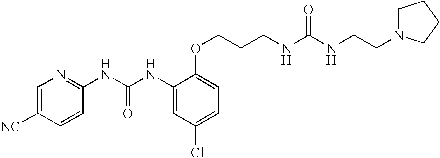

1-[2-(3-Amino-propoxy)-5-chloro-phenyl]-3-(5-cyano-pyridin-2-yl)-urea (100 mg, 0.262 mmol) was suspended in tetrahydrofuran (4 mL) over potassium carbonate (88 mg, 0.640 mmol). 1-Chloro-2-isocyanato-ethane (21 μL, 0.288 mmol) was added and the reaction was stirred at room temperature for 2 hours. At this time, LCMS indicated complete consumption of starting material to give the desired urea. The crude reaction mixture was then partitioned into three equal portions, and to the first portion a solution of pyrrolidine (22 μL, 0.262 mmol) and triethylamine (37 μL, 0.262 mmol) was added. The other portions were similarly reacted with other amines. Each portion was stirred at room temperature overnight, at which point LCMS indicated disappearance of starting chloride and the presence of the desired amine. The potassium carbonate was removed by filtration and the solvent removed in vacuo with the aid of toluene. The resulting tan solid was triturated in ethyl acetate and the solvent was removed by filtration to give the title compound as a white solid. LCMS: Method A, Rₜ=1.28 min, [MH⁺=486.2].

### Example 21

### 1-(5-Chloro-2-{3-[3-(2-diethylamino-ethyl)-ureido]-propoxy}-phenyl)-3-(5-cyano-pyridin-2-yl)-urea (Compound I-99)

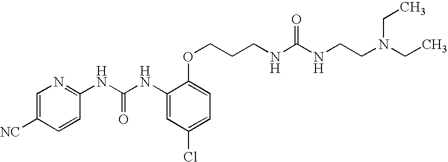

The title compound was prepared from the appropriate reagents, using a procedure similar to that described in Example 20. LCMS: Method A, Rₜ=1.35 min, [MH⁺=488.2].

### Example 22

### 1-(5-Chloro-2-{3-[3-(2-methylamino-ethyl)-ureido]-propoxy}-phenyl)-3-(5-cyano-pyridin-2-yl)-urea (Compound I-100)

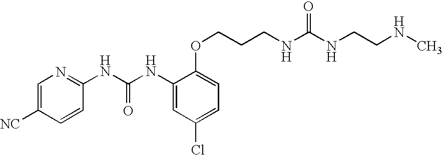

The title compound was prepared from the appropriate reagents, using a procedure similar to that described in Example 20. LCMS: Method A, Rₜ=1.24 nin, [MH⁺=446.9].

### Example 23

### 1-{5-Chloro-2-[3-(3-cyclohexyl-ureido)-propoxy]-phenyl-3-(5-cyano-pyridin-2-yl)-urea (Compound I-101)

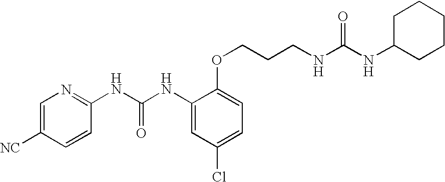

The title compound was prepared from the appropriate reagents, using a procedure similar to that described in Example 20. LCMS: Method A, Rₜ=2.13 min, [MH⁺=471.2].

### Example 24

### 1-{2-[3-(3-Benzyl-ureido)-propoxy]-5-chloro-phenyl-3-(5-cyano-pyridin-2-yl)-urea (Compound I-102)

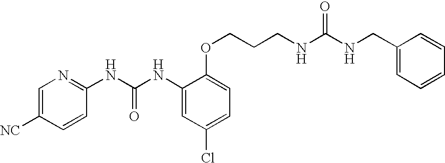

The title compound was prepared from the appropriate reagents, using a procedure similar to that described in Example 20. LCMS: Method A, Rₜ=2.02 min, [MH⁺=479.2].

### Example 25

### 1-{5-Chloro-2-[3-(3-thiophen-3-yl-ureido)-propoxy]-phenyl-3-(5-cyano-pyridin-2-yl)-urea (Compound I-103)

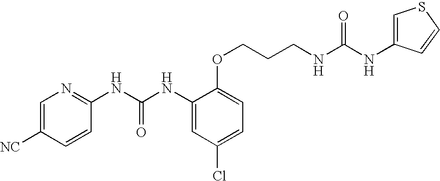

The title compound was prepared from the appropriate reagents, using a procedure similar to that described in Example 20. LCMS: Method A, Rₜ=1.92 min, [MH⁺=471.2].

### Example 26

### 1-(5-Chloro-2-{3-[3-(4-dimethylamino-phenyl)-ureido]-propoxy-phenyl)-3-(5-cyano-pyridin-2-yl)-urea (Compound I-104)

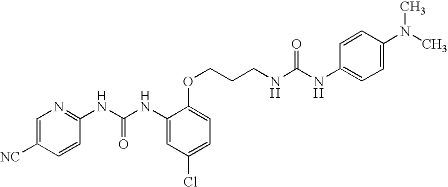

The title compound was prepared from the appropriate reagents, using a procedure similar to that described in Example 20. LCMS: Method A, Rₜ=1.42 min, [MH⁺=508.2].

### Example 27

### (3-{4-Chloro-2-[3-(5-cyano-pyridin-2-yl)-ureido]-phenoxy}-propyl)-carbamic acid 2-dimethylamino-ethyl ester (Compound I-59)

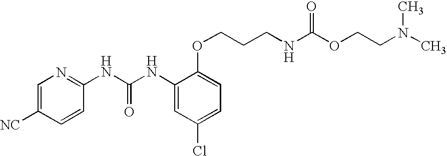

N,N-Dimethylaminoethanol (13 μL, 0.131 mmol) was slowly added to a pre-stirred solution of triphosgene (43 mg, 0.144 mmol) in methylene chloride (2 mL) over excess sodium bicarbonate at 0° C. This was stirred for 1.5 h at room temperature until gas evolution had ceased, at which time solid potassium carbonate (88 mg, 0.640 mmol) was added, followed by 1-[2-(3-amino-propoxy)-5-chloro-phenyl]-3-(5-cyano-pyridin-2-yl)-urea (50 mg, 0.131 mmol) in methylene chloride (1 mL). This mixture was allowed to stir at room temperature for 3 days, at which point LCMS indicated disappearance of starting material and conversion to the desired product. The solids were removed by filtration, and the solvent was removed in vacuo. The resulting tan solid was triturated in ethyl acetate and the solvent was removed by filtration to give the title compound as a white solid. LCMS: Method A, Rₜ=1.48 min. [MH⁺=461.2].

### Example 28

### (3-{4-Chloro-2-[3-(5-cyano-pyridin-2-yl)-ureido]-phenoxy-propyl)-carbamic acid 2-morpholin-4-yl-ethyl ester (Compound I-60)

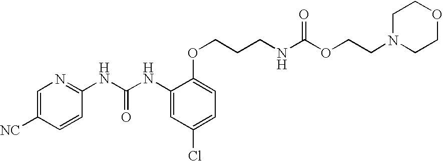

The title compound was prepared from the appropriate reagents, using a procedure similar to that described in Example 27. LCMS: Method A, Rₜ=1.23 min, [MH⁺=503.1].

### Example 29

### (S)-2-Amino-N-(3-{4-chloro-2-[3-(5-cyano-pyridin-2-yl)-ureido]-phenoxy}-propyl)-3-(1H-imidazol-4-yl)-propionamide (Compound I-106)

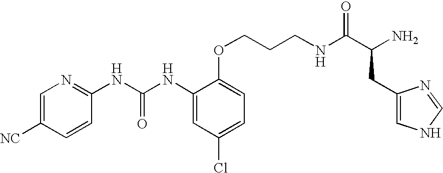

O-(7-Azabenzotriazol-1-yl)-N,N,N′,N′-tetramethyluronium hexafluorophosphate (HATU) (100 mg, 0.262 mmol) was added to a solution of 4-(2-tert-butoxycarbonylamino-2-carboxy-ethyl)-imidazole-1-carboxylic acid tert-butyl ester (93 mg, 0.262 mmol) and triethylamine (183 μL, 1.31 mmol) in methylene chloride (3 mL). This mixture was stirred for 5 minutes at room temperature, at which time a solution of 1-[2-(3-amino-propoxy)-5-chloro-phenyl]-3-(5-cyano-pyridin-2-yl)-urea (50 mg, 0.131 mmol) and triethylamine (183 μL, 1.31 mmol) in methylene chloride (1 mL) was added. This mixture was stirred at room temperature for one hour, at which time LCMS indicated disappearance of starting amine and the presence of the desired amide. Ethyl acetate (20 mL) was added and the reaction mixture was filtered through a short plug of silica. The crude product was then treated with hydrochloric acid in dioxane (0.66 mL, 2.6 mmol) and stirred at room temperature overnight. LCMS indicated disappearance of both Boc protecting groups. The resulting white precipitate was collected, rinsed with ethyl acetate and dried to give the bis hydrochloride salt of the desired compound as a white solid. LCMS: Method A, Rₜ=1.98 min, [MH⁺=483.2].

### Example 30

### (S)-2-Amino-3-(4-amino-phenyl)-N-(3-{4-chloro-2-[3-(S-cyano-pyridin-2-yl)-ureido]-phenoxy-propyl)-propionamide (Compound I-107)

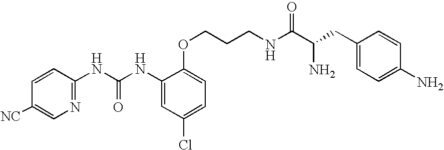

The title compound was prepared from the appropriate reagents, using a procedure similar to that described in Example 29. LCMS: Method A, Rₜ=1.20 min, [MH⁺=508.2].

### Example 31

### (S)-1,2,3,4-Tetrahydro-isoquinoline-3-carboxylic acid (3-{4-chloro-2-[3-(5-cyano-pyridin-2-yl)-ureido]-phenoxy-propyl)-amide (Compound I-108)

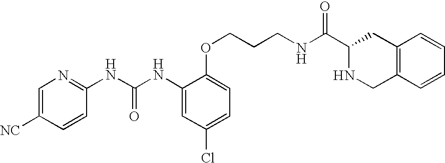

The title compound was prepared from the appropriate reagents, using a procedure similar to that described in Example 29. LCMS: Method A, Rₜ=1.31 min, [MH⁺=505.1].

### Example 32

### (S)-Pyrrolidine-2-carboxylic acid (3-{4-chloro-2-[3-(5-cyano-pyridin-2-yl)-ureido]-phenoxy-propyl)-amide (Compound I-109)

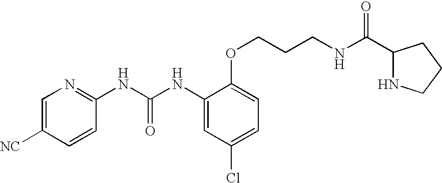

The title compound was prepared from the appropriate reagents, using a procedure similar to that described in Example 29. LCMS: Method A, Rₜ=1.20 min, [MH⁺=443.2].

### Example 33

### General Procedures

LCMS Method A

Samples were analysed using a Waters Symmetry [C8, 50×4.6 mm, 3.5 uM] eluting with acetonitrile/water/0.1% formic acid (5-95% acetonitrile) for 5 minutes with a flow rate of 1.5 mL/min.

LCMS Method B

Samples were analysed using a Waters Symmetry [C8, 50×4.6 mm, 3.5 uM] eluting with methanol/water/10 mM Ammonium acetate (5-95% methanol) for 5 minutes with a flow rate of 1.5 mL/min.

NMR

NMR data was recorded on a Bruker NMR spectrometer (400 MHz).

### Example 34

### Chk-1 Assays

Chk-1 Expression & Purification:

Recombinant human Chk-1 was expressed as a fusion protein with glutathione S-transferase at the amino-terminus (GST-Chk-1) using standard baculovirus vectors and (Bac-to-Bac®) insect cell expression system purchased from GIBCO™ Invitrogen.

Recombinant protein expressed in insect cells was purified using glutathione sepharose (Amersham Biotech) using standard procedures described by the manufacturer.

Chk-1 FlashPlate® Kinase Assay:

Assays (25 μL) contained 8.7 nM GST-Chk-1, 10 mM MES, 0.1 mM ethylene glycol-bis(β-aminoethylether)-N,N,N′,N′-tetraacetic acid (EGTA, pH 8.0), 2 mM DTT, 0.05% Tween 20, 3 μM peptide substrate (Biotin-ILSRRPSYRKILND-free acid) (SEQ ID NO: 1), 1 μM ATP, 0.4 μCi ³³P-γ-ATP (NEN), 4% DMSO. Reactions were incubated for 30 minutes at room temperature, terminated with 50 μL of 50 mM EDTA and 90 μL were transferred to streptavidin-coated FlashPlates® (NEN) and incubated for 1 hour at room temperature. Plates were washed with phosphate buffered saline containing 0.01% Tween-20 and 10 mM sodium pyrophosphate. Plates were dried, sealed with Topseal™ (NEN) and amount of ³³P incorporated into the peptide substrate measure using a Packard Topcount® NXT™ scintillation counter with standard settings.

In this assay, compounds of the present invention inhibited Chk-1 induced ³³P incorporation at a concentration of 10 μM. For example, in this assay a number of compounds provided 50% or better inhibition at a concentration of 10 μM. Examples of such compounds include compounds I-2,1-4, I-5, I-6, I-7, I-8, I-9, I-10, I-11, I-12, I-13, I-14, I-15, I-16, I-18, I-20, I-39, I-110, and I-111.

Certain compounds tested also provided 50% or better inhibition of Chk-1 at a concentration of 1.0 μM. Examples of such compounds include I-2, I-4, I-5, I-6, I-7, I-8, I-9, I-11, I-12, I-13, I-14, I-15, I-16, I-18, I-39, and I-111.

Compounds of the present invention can also be tested in DELFIA®, fluorescence polarization, and SPA filtration assays.

Chk-1 DELFIA® Kinase Assay:

Assays (25 μL) utilize 6.4 nM GST-Chk-1 containing 25 mM Tris, pH 8.5, 20% glycerol, 50 mM sodium chloride (NaCl), 0.1% Surfact-Amps® 20, 1 μM peptide substrate (Biotin-GLYRSPSMPEN-amide) (SEQ ID NO: 3), 2 mM DTT, 4% DMSO, 12.5 μM ATP, 5 mM MgCl₂ and are reacted for 30 minutes at room temperature. Reactions are terminated with 100 μL of Stop buffer containing 1% BSA, 10 mM Tris, pH 8.0, 150 mM NaCl, 100 mM EDTA. Stopped reactions (100 μL) are transferred to 96 well neutravidin plates (Pierce) to capture the biotin-peptide substrate during a 30 minute room temperature incubation. Wells are washed and reacted with 100 μL PerkinElmer Wallac Assay Buffer containing 21.5 ng/mL anti-phospho-Ser216-Cdc25c rabbit polyclonal antibody from Cell Signaling Technology (Beverly, Mass.) and 292 ng/mL europium labeled anti-rabbit-IgG for 1 hour at room temperature. Wells are washed and europium released from the bound antibody by addition of Enhancement Solution (100 μL) (PerkinElmer Wallac) and detected using a Wallac Victor2™ using standard manufacturer settings.

Chk-1 Fluorescence Polarization Assays:

Assays utilize 10 nM GST-Chk-1 and contain 5 mM 2-(N-morpholino)ethanesulfonic acid (MES, pH 6.5), 5 mM magnesium chloride (MgCl₂), 0.05% Tween®-20, 1 μM adenosine 5′ triphosphate (ATP), 2 mM 1,4-Dithio-DL-threitol (DTT), 1 μM peptide substrate (Biotin-ILSRRPSYRKILND-free acid) (SEQ ID NO: 1), 10 nM peptide substrate tracer (Fluorescine-GSRRP-pS-YRKI-free acid) (pS=phosphorylated-Serine) (SEQ ID NO: 2), 60 ng anti-phospho-CREB(S133) mouse monoclonal IgG purified on Protein G sepharose from crude mouse ascites purchased from Cell Signaling Technologies (Beverly, Mass.), 4% dimethyl sulfoxide (DMSO) and 30 μM inhibitor. Reactions are incubated at room temperature for 140 minutes and are terminated by addition of 25 mM EDTA (pH 8.0). Stopped reactions are incubated for 120 minutes at room temperature and fluorescence polarization values are determined using a Molecular Devices/LJL Biosystems Analyst™ AD (Sunnyvale, Calif.) with standard fluorescine settings.

Chk-1 SPA Filtration Assay:

Assays (25 μL) contain 10 nM GST-Chk-1, 10 mM MES, 2 mM DTT, 10 mM MgCl₂, 0.025% Tween®-20, 1 μM peptide substrate (Biotin-ILSRRPSYRKILND-free acid) (SEQ ID NO: 1), 1 μM ATP, 0.1 μCi ³³P-γ-ATP (New England Nuclear, NEN) and are reacted for 90 minutes at room temperature. Reactions are terminated by adding 55 μL of phosphate buffered saline containing 50 mM EDTA, 6.9 mM ATP, 0.5 mg Scintillation proximity assay (SPA) beads (Amersham Biosciences). Peptide substrate is allowed to bind beads for 10 minutes at room temperature followed by filtration on a Packard GF/B Unifilter plate and washing with phosphate buffered saline. Dried plates are sealed with Topseal™ (NEN) and ³³P incorporated to peptide substrate is detected using a Packard Topcount® scintillation counter with standard settings for ³³P.

### Example 35

### WST Cytotoxicity Assay

HT29, HCT116 (5000 cells/well) or other cells were seeded (75 μL) to 96 well clear bottom plates at densities which provide linear growth curves for 72 hours. Cells were cultured under sterile conditions in appropriate media and for HT29 & HCT116 this media was McCoy's 5A containing 10% Fetal Bovine Serum (FBS). Following the initial seeding of cells they are incubated at 37° C., 5% CO₂ from 17 to 24 hours at which time the appropriate DNA damaging agents (camptothecins, 5-fluorouracil, doxorubicin, and etoposide) are added at increasing concentrations to a point which is capable of causing at least 80% cell killing with in 48 hours. Final volume of all DNA damaging agent & compound additions was 25 μL and assays contained <1% DMSO final. At the same time as DNA damaging agent addition, Chk-1 inhibitor was added at fixed concentrations to each DNA damaging agent titration to observe enhancement of cell killing. In addition, toxicity of each Chk-1 inhibitor alone was observed. By doing this over a range of Chk-1 inhibitor concentrations, compounds were identified which maximally enhance (2-30 fold) cell killing by each DNA damaging agent and generated ≦80% cell killing by the compound alone. Cell viability/cell killing under the conditions described above was determined by addition WST reagent (Roche) according to the manufacturer at 47 hours following DNA damage & Chk-1 inhibitor addition and following a 3.5 hour or 2.5 hour incubation at 37 C., 5% CO₂, OD₄₅₀ was measured.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention and appended claims.

1. A compound of formula (I): 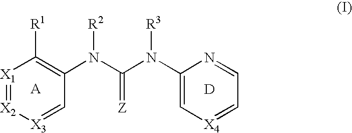
or a pharmaceutically acceptable salt thereof, wherein:
X₁-X₃ are independently CH or N, provided that X₁-X₃ are not all N;
X₄ is CH or N;
Z is O, S, N—R, or N—CN;
Ring A is optionally substituted at any substitutable carbon by R⁴;
Ring D is optionally substituted by C₁₋₄ aliphatic or haloaliphatic, —OR⁷, —SR⁷, —C(O)R, —CO₂R⁷, —SO₂R, —CN, —C(O)N(R⁷)₂, —N(R⁷)C(O)R, or —N(R⁷)₂, and is optionally fused to an optionally substituted phenyl or optionally substituted cyclohexyl ring;
R¹ is -T-W or -V-T-W;
T is a C₁₋₆ straight or branched alkylidene chain that is optionally substituted by F, —OR⁶, —N(R⁶)₂₁ or —CO₂R⁶, and is optionally interrupted by —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—, wherein the alkylidene chain or a portion thereof is optionally part of a 3-6 membered ring system;
V is —O—, —S—, —N(R⁵)—, —S(O)—, —SO₂—, —C(O)—, —OC(O)—, —N(R⁵)C(O)—, —C(O)N(R⁵)—, —SO₂N(R⁵)—, or —N(R⁵)SO₂—;
W is —C(O)N(R⁹)₂, —N(R⁶)—C(O)—R¹⁰, —N(R⁶)—C(O)—N(R⁹)₂, —N(R⁶)—C(O)—OR¹¹, —O—C(O)—N(R⁹)₂, —NH—C(═NH)—R¹⁰, or —NH—(═NH)—NH—R⁹;
each of R² and R³ independently is hydrogen or C₁₋₆ alkyl optionally substituted with —N(R⁸)₂, —C(O)R, —CO₂R, or SO₂R; or R² and R³, taken together with the intervening atoms, form an optionally substituted 5-6 membered ring;
each R⁴ independently is halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, or an optionally substituted C₁₋₈ aliphatic, aryl, aralkyl, heterocyclyl, heterocyclylalkyl, heteroaryl, or heteroaralkyl group; or two adjacent R⁴, taken together, form an optionally substituted phenyl, pyridyl or heterocyclyl ring fused to Ring A;
each R⁵ independently is hydrogen, C₁₋₆ aliphatic, —CO₂R, —SO₂R, or —C(O)R; or —N(R⁵)₂ is an optionally substituted nitrogen-containing heterocyclyl;
each R⁶ independently is hydrogen or an optionally substituted C₁₋₃ aliphatic;
each R⁷ independently is hydrogen or an optionally substituted C₁₋₃ aliphatic; or —N(R⁷)₂ is an optionally substituted nitrogen-containing heterocyclyl;
each R⁸ independently is a C₁₋₃ alkyl; or —N(R⁸)₂ is an optionally substituted nitrogen-containing heterocyclyl;
each R⁹ independently is hydrogen, an optionally substituted C₁₋₆ aliphatic, an optionally substituted heterocyclyl, or an optionally substituted heteroaryl; or —N(R⁹)₂ is an optionally substituted nitrogen-containing heterocyclyl;
each R¹⁰ independently is an optionally substituted aryl, heteroaryl, heterocyclyl, or C₁₋₆ aliphatic group;
R¹¹ is an optionally substituted aryl, heteroaryl, heterocyclyl, or C₁₋₆ aliphatic, group, provided that R¹¹ is other than tert-butyl or arylmethyl; and
each R independently is hydrogen or an optionally substituted C₁₋₆ aliphatic, aryl, aralkyl, heteroaryl, or heteroaralkyl group.

2. The compound of claim 1, wherein Ring D has the formula 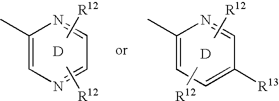
where:
each R¹² is independently selected from hydrogen, C₁₋₄ aliphatic or haloaliphatic, —OR⁷, —SR⁷, —C(O)R, —CO₂R⁷, —SO₂R, —CN, —C(O)N(R⁷)₂, —N(R⁷)C(O)R, or —N—(R⁷)₂, or two adjacent R¹² together form an optionally substituted fused phenyl or cyclohexyl ring; and
R¹³ is —CO₂R⁷, —CN, or —C(O)N(R⁷)₂.

3. The compound of claim 2, wherein Ring D is selected from the group consisting of 2-pyrazinyl, 5-methyl-pyrazin-2-yl, 5-cyano-pyrazin-2-yl, 5-cyano-pyridin-2-yl, 5-carbamoyl-pyridin-2-yl, and 5-carbamoyl-pyrazin-2-yl.

4. The compound of claim 1, wherein Z is O, and R² and R³ are each hydrogen.

5. The compound of claim 1, wherein V is —O— and T is a straight or branched C₂₋₅alkylidene chain.

6. The compound of claim 1, wherein:
R² and R³ are each hydrogen;
Z is oxygen;
each of X₁-X₃ is CH;
V is —O—, and T is a straight or branched C₂₋₅ alkylidene chain; and
W is —C(O)N(R⁹)₂, —NH—C(O)—R¹⁰, —NH—C(O)—N(R⁹)₂, —NH—C(O)—OR¹¹, —O—C(O)—N(R⁹)₂, —NH—C(═NH)—R¹⁰, or —NH—(═NH)—NH—R⁹.

7. The compound of claim 5, wherein W is —C(O)N(R⁹)₂ or —N(R⁶)—C(O)—N(R⁹)₂, and —N(R⁹)₂ is an optionally substituted nitrogen-containing heterocyclyl selected from the group consisting of morpholinyl, piperidinyl, piperazinyl, thiomorpholinyl, and pyrrolidinyl.

8. The compound of claim 6, wherein —N(R⁹)₂ is morpholinyl or 4-methylpiperazinyl.

9. The compound of claim 6, wherein W is —C(O)N(R⁹)₂ or —NH—C(O)—N(R⁹)₂.

10. The compound of claim 1, wherein W is —N(R⁶)—C(O)—R¹⁰ and R¹⁰ is a C₁₋₆ aliphatic, which optionally is substituted by 1 to 3 groups independently selected from -fluoro, —OR, CN, —CO₂R⁷, —N(R⁷)₂, —NH—C(═NH)—NH₂, or an optionally substituted aryl, cycloaliphatic, heteroaryl, or heterocyclyl group.

11. The compound of claim 10, wherein W has the formula —NH—C(O)—(CH₂)ₘ—CH(N(R¹³)₂)—(CH₂)ₙ—Y¹, —NH—C(O)—CH(CH₂—N(R¹³)₂)—(CH₂)ₙ—Y¹, or —NH—C(O)—(CH₂)ₙ—Y¹, where R¹³ is hydrogen or C₁₋₃ aliphatic, m is 0 or 1, n is 0-3, and Y¹ is an optionally substituted aryl, cycloaliphatic, heteroaryl, or heterocyclyl group.

12. The compound of claim 11, wherein Y¹ is C₆₋₁₀ aryl optionally substituted by one to three substituents independently selected from the group consisting of -halo, —OR, —SR, —CN, —NO₂, —N(R⁵)₂, —N(R⁵)C(O)R, —N(R⁵)CO₂R, —N(R⁵)C(O)N(R⁵)₂, —C(O)N(R⁵)₂, —C(O)R⁵, —OC(O)N(R⁵)₂, —CO₂R, —SO₂R, —S(O)R, —SO₂N(R⁵)₂, —N(R⁵)SO₂R, and optionally substituted C₁₋₈ aliphatic, aryl, aralkyl, heterocyclyl, heterocyclylalkyl, heteroaryl, and heteroaralkyl groups.

13. The compound of claim 10, wherein W has the formula —NH—C(O)—(CH₂)ₘ—CH(N(R¹³)₂)—Y², where R¹³ is hydrogen or C₁₋₃ aliphatic, m is 0 or 1, and Y² is a C₁₋₆ aliphatic, which optionally is substituted by one or two groups selected from the group consisting of —OR, —CN, —CO₂R⁷, —N(R⁷)₂, —NH—C(═NH)—NH₂, and —NH—C(═NH)—R.

14. The compound of claim 1, wherein Ring A has the formula:

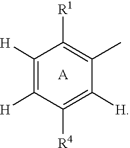

15. The compound of claim 14, wherein:
R⁴ is selected from the group consisting of —F, —Cl, —Br, —I, —COOR^{a}, —NHCOR^{a}, —CF₃, —CH₃, —CH₂CH₃, —CH(CH₃)₂, —N(R^{a})₂, —CH₂N(R^{a})₂, —CH₂CH₂N(R^{a})₂, piperidinyl, morpholinyl and pyrrolidinyl; and
R^{a} is —CH₃, —CH₂CH₃, —CH₂CH₂NH₂, —CH₂CH₂NH(CH₃), —CH₂CH₂N(CH₃)₂, —CH₂CH₂(N-morpholinyl), —CH₂CH₂(N-piperidinyl) or —CH₂CH₂(N-pyrrolidinyl).

16. A compound selected from the group consisting of:
2-Amino-N-(3-{4-chloro-2-[3-(5-cyano-pyridin-2-yl)-ureido]-phenoxy}-propyl)-3-(4-chloro-phenyl)-propionamide;
6-[3-(2-{3-[2-Amino-3-(4-chloro-phenyl)-propionylamino]-propoxy}-5-chloro-phenyl)-ureido]-nicotinamide;
2-Amino-N-(3-{4-chloro-2-[3-(5-methyl-pyrazin-2-yl)-ureido]-phenoxy}-propyl)-2-(4-chloro-phenyl)-acetamide;
N-(3-{4-Chloro-2-[3-(5-methyl-pyrazin-2-yl)-ureido]-phenoxy}-propyl)-2-dimethylamino-acetamide;
Morpholine-4-carboxylic acid (3-{4-chloro-2-[3-(5-methyl-pyrazin-2-yl)-ureido]-phenoxy}-propyl)-amide;
N-(3-{4-Chloro-2-[3-(5-methyl-pyrazin-2-yl)-ureido]-phenoxy}-propyl)-2-morpholin-4-yl-acetamide;
N-(3-{4-Chloro-2-[3-(5-methyl-pyrazin-2-yl)-ureido]-phenoxy}-propyl)-2-(4-methyl-piperazin-1-yl)-acetamide;
1-[5-Chloro-2-(2-morpholin-4-yl-2-oxo-ethoxy)-phenyl]-3-(5-methyl-pyrazin-2-yl)-urea;
4-[3-(4-Chloro-benzoylamino)-propoxy]-N-(2-dimethylamino-ethyl)-3-[3-(5-methyl-pyrazin-2-yl)-ureido]-benzamide;
4-Chloro-N-(3-{2-[3-(5-methyl-pyrazin-2-yl)-ureido]-5-pyrrolidin-1-yl-phenoxy}-propyl)-benzamide;
2-Dimethylamino-N-(3-{2-[3-(5-methyl-pyrazin-2-yl)-ureido]-5-pyrrolidin-1-yl-phenoxy}-propyl)-acetamide;
Morpholine-4-carboxylic acid (3-{4-chloro-2-[3-(5-cyano-pyridin-2-yl)-ureido]-phenoxy}-propyl)-amide;
4-Chloro-N-(3-{5-(4-methyl-piperazin-1-yl)-2-[3-(5-methyl-pyrazin-2-yl)-ureido]-phenoxy}-propyl)-benzamide;
1-{5-Chloro-2-[4-(4-methyl-piperazin-1-yl)-4-oxo-butoxy]-phenyl}-3-(5-cyano-pyridin-2-yl)-urea;
N-(3-{4-Chloro-2-[3-(5-methyl-pyrazin-2-yl)-ureido]-phenoxy}-propyl)-4-dimethylamino-benzamide;
2-Amino-N-(3-{4-chloro-2-[3-(5-methyl-pyrazin-2-yl)-ureido]-phenoxy}-propyl)-3-(4-chloro-phenyl)-propionamide;
N-(3-{4-Chloro-2-[3-(5-cyano-pyridin-2-yl)-ureido]-phenoxy}-propyl)-2-dimethylamino-acetamide;
4-Methyl-piperazine-1-carboxylic acid (3-{4-chloro-2-[3-(5-cyano-pyridin-2-yl)-ureido]-phenoxy}-propyl)-amide;
4-Chloro-N-(3-{4-chloro-2-[3-(5-methyl-pyrazin-2-yl)-ureido]-phenoxy}-propyl)-benzamide;
4-Chloro-N-(3-{4-(4-methyl-piperazine-1-carbonyl)-2-[3-(5-methyl-pyrazin-2-yl)-ureido]-phenoxy}-propyl)-benzamide;
N-(3-{4-Chloro-2-[3-(5-cyano-pyridin-2-yl)-ureido]-phenoxy}-propyl)-3-piperidin-1-yl-propionamide;
N-(3-{4-Chloro-2-[3-(5-methyl-pyrazin-2-yl)-ureido]-phenoxy}-propyl)-3diethylamino-propionamide;
3-Acetylamino-N-(3-{4-chloro-2-[3-(5-cyano-pyridin-2-yl)-ureido]-phenoxy}-propyl)-propionamide;
N-(3-{4-Chloro-2-[3-(5-methyl-pyrazin-2-yl)-ureido]-phenoxy}-propyl)-4-dimethylamino-butyramide;
N-(3-{4-Chloro-2-[3-(5-cyano-pyridin-2-yl)-ureido]-phenoxy}-propyl)-2-pyrrolidin-1-yl-acetamide;
N-(3-{4-Chloro-2-[3-(5-methyl-pyrazin-2-yl)-ureido]-phenoxy}-propyl)-3-phenylamino-propionamide;
2-(4-Acetyl-piperazin-1-yl)-N-(3-{4-chloro-2-[3-(5-cyano-pyridin-2-yl)-ureido]-phenoxy}-propyl)-acetamide;
N-(3-{4-Chloro-2-[3-(5-methyl-pyrazin-2-yl)-ureido]-phenoxy}-propyl)-2-piperazin-1-yl-acetamide;
N-(3-{4-Chloro-2-[3-(5-cyano-pyridin-2-yl)-ureido]-phenoxy}-propyl)-2-(3-chloro-phenyl)-acetamide;
N-(3-{4-Chloro-2-[3-(5-cyano-pyridin-2-yl)-ureido]-phenoxy}-propyl)-4-(4-methoxy-phenyl)-butyramide;
N-(3-{4-Chloro-2-[3-(5-cyano-pyridin-2-yl)-ureido]-phenoxy}-propyl)-4-fluoro-benzamide;
N-(3-{4-Chloro-2-[3-(5-cyano-pyridin-2-yl)-ureido]-phenoxy}-propyl)-2-(2-cyano-phenyl)-acetamide;
N-(3-{4-Chloro-2-[3-(5-cyano-pyridin-2-yl)-ureido]-phenoxy}-propyl)-3-methyl-benzamide;
4-Bromo-N-(3-{4-chloro-2-[3-(5-methyl-pyrazin-2-yl)-ureido]-phenoxy}-propyl)-benzamide;
N-(3-{4-Chloro-2-[3-(5-methyl-pyrazin-2-yl)-ureido]-phenoxy}-propyl)-2-fluoro-6-trifluoromethyl-benzaminde;
N-(3-{4-Chloro-2-[3-(5-methyl-pyrazin-2-yl)-ureido]-phenoxy}-propyl)-4-trifluoromethoxy-benzamide;
N-(3-{4-Chloro-2-[3-(5-cyano-pyridin-2-yl)-ureido]-phenoxy}-propyl)-2-pyridin-3-yl-acetamide;
1,2,3,4-Tetrahydro-quinoline-6-carboxylic acid (3-{4-chloro-2-[3-(5-methyl-pyrazin-2-yl)-ureido]-phenoxy}-propyl)-amide;
N-(3-{4-Chloro-2-[3-(5-cyano-pyridin-2-yl)-ureido]-phenoxy}-propyl)-2-dimethylamino-acetamide;
N-(3-{4-Chloro-2-[3-(5-methyl-pyrazin-2-yl)-ureido]-phenoxy}-propyl)-3-dimethylamino-propionamide;
N-(3-{4-Chloro-2-[3-(5-methyl-pyrazin-2-yl)-ureido]-phenoxy}-propyl)-2-cyclohexyl-acetamide;
N-(3-{4-Chloro-2-[3-(5-methyl-pyrazin-2-yl)-ureido]-phenoxy}-propyl)-3-methoxy-propionamide;
N-(3-{4-Chloro-2-[3-(5-cyano-pyridin-2-yl)-ureido]-phenoxy}-propyl)-2-thiophen-3-yl-acetamide;
N-(3-{4-Chloro-2-[3-(5-methyl-pyrazin-2-yl)-ureido]-phenoxy}-propyl)-succinamic acid methyl ester;
N-(3-{4-Chloro-2-[3-(5-cyano-pyridin-2-yl)-ureido]-phenoxy}-propyl)-4-(1H-indol-3-yl)-butyramide;
N-(3-{4-Chloro-2-[3-(5-methyl-pyrazin-2-yl)-ureido]-phenoxy}-propyl)-3-piperidin-1-yl-propionamide;
N-(3-{4-Chloro-2-[3-(5-methyl-pyrazin-2-yl)-ureido]-phenoxy}-propyl)-3-(1H-imidazol-4-yl)-propionamide;
2-Amino-5-guanidino-pentanoic acid (3-{4-chloro-2-[3-(5-methyl-pyrazin-2-yl)-ureido]-phenoxy}-propyl)-amide;
3-(4-Amino-phenyl)-N-(3-{4-chloro-2-[3-(5-cyano-pyridin-2-yl)-ureido]-phenoxy}-propyl)-2-methylamino-propionamide;
2-Amino-N-(3-{4-chloro-2-[3-(5-cyano-pyridin-2-yl)-ureido]-phenoxy}-propyl)-4-phenyl-butyramide;
2-Amino-N-(3-{4-chloro-2-[3-(5-cyano-pyridin-2-yl)-ureido]-phenoxy}-propyl)-3-(2-cyano-phenyl)-propionamide;
2-Amino-N-(3-{4-chloro-2-[3-(5-cyano-pyridin-2-yl)-ureido]-phenoxy}-propyl)-3-o-tolyl-propionamide;
2-Amino-N-(3-{4-chloro-2-[3-(5-cyano-pyridin-2-yl)-ureido]-phenoxy}-propyl)-3-(2,6-dichloro-phenyl)-propionamide;
2-Amino-N-(3-{4-chloro-2-[3-(5-cyano-pyridin-2-yl)-ureido]-phenoxy}-propyl)-3-(4-nitro-phenyl)-propionamide;
2-Amino-N-(3-{4-chloro-2-[3-(5-cyano-pyridin-2-yl)-ureido]-phenoxy}-propyl)-3-(3,4-difluoro-phenyl)-propionamide;
2-Amino-N-(3-{4-chloro-2-[3-(5-cyano-pyridin-2-yl)-ureido]-phenoxy}-propyl)-3-(4-hydroxy-3-methoxy-phenyl)-propionamide;
2-Amino-N-(3-{4-chloro-2-[3-(5-cyano-pyridin-2-yl)-ureido]-phenoxy}-propyl)-3-(2-trifluoromethyl-phenyl)-propionamide;
3-(4-Bromo-phenyl)-N-(3-{4-chloro-2-[3-(5-cyano-pyridin-2-yl)-ureido]-phenoxy}-propyl)-2-methylamino-propionamide;
(3-{4-Chloro-2-[3-(5-cyano-pyridin-2-yl)-ureido]-phenoxy}-propyl)-carbamic acid 2-dimethylamino-ethyl ester;
(3-{4-Chloro-2-[3-(5-cyano-pyridin-2-yl)-ureido]-phenoxy}-propyl)-carbamic acid 2-morpholin-4-yl-ethyl ester;
(3-{4-Chloro-2-[3-(5-cyano-pyridin-2-yl)-ureido]-phenoxy}-propyl)-carbamic acid isobutyl ester;
(3-{4-Chloro-2-[3-(5-methyl-pyrazin-2-yl)-ureido]-phenoxy}-propyl)-carbamic acid 2-hydroxy-ethyl ester;
(3-{4-Chloro-2-[3-(5-methyl-pyrazin-2-yl)-ureido]-phenoxy}-propyl)-carbamic acid cyclohexylmethyl ester;
(3-{4-Chloro-2-[3-(5-methyl-pyrazin-2-yl)-ureido]-phenoxy}-propyl)-carbamic acid propyl ester;
(3-{4-Chloro-2-[3-(5-methyl-pyrazin-2-yl)-ureido]-phenoxy}-propyl)-carbamic acid 2-cyclopentyl-ethyl ester;
(3-{4-Chloro-2-[3-(5-methyl-pyrazin-2-yl)-ureido]-phenoxy}-propyl)-carbamic acid cycloheptylmethyl ester;
(3-{4-Chloro-2-[3-(5-methyl-pyrazin-2-yl)-ureido]-phenoxy}-propyl)-carbamic acid piperidin-4-ylmethyl ester;
(3-{4-Chloro-2-[3-(5-methyl-pyrazin-2-yl)-ureido]-phenoxy}-propyl)-carbamic acid 2-pyrrolidin-1-yl-ethyl ester;
(3-{4-Chloro-2-[3-(5-methyl-pyrazin-2-yl)-ureido]-phenoxy}-propyl)-carbamic acid 2-morpholin-4-yl-ethyl ester;
(3-{4-Chloro-2-[3-(5-cyano-pyridin-2-yl)-ureido]-phenoxy}-propyl)-carbamic acid 3-methylamino-propyl ester;
{3-[4-Chloro-2-(3-pyridin-2-yl-ureido)-phenoxy]-propyl}-carbamic acid 4-amino-butyl ester;
(3-{4-Chloro-2-[3-(5-methyl-pyrazin-2-yl)-ureido]-phenoxy}-propyl)-carbamic acid 2-(1H-imidazol-4-yl)-ethyl ester;
1-{5-Chloro-2-[3-(3-furan-2-ylmethyl-ureido)-propoxy]-phenyl}-3-(5-cyano-pyridin-2-yl)-urea;
1-(2-[3-{3-Benzo[1,3]dioxol-5-yl-ureido)-propoxy]-5-chloro-phenyl}-3-(5-cyano-pyridin-2-yl)-urea;
1-(5-Chloro-2-{3-[3-(2-thiophen-3-yl-ethyl)-ureido]-propoxy}-phenyl)-3-(5-cyano-pyridin-2-yl)-urea;
3-[3-(3-{4-Chloro-2-[3-(5-cyano-pyridin-2-yl)-ureido]-phenoxy}-propyl)-ureido]propionic acid methyl ester;
1-{5-Chloro-2-[3-(3,3-dimethyl-ureido)-propoxy]-phenyl}-3-(5-cyano-pyridin-2-yl)-urea;
Pyrrolidine-1-carboxylic acid (3-{4-chloro-2-[3-(5-cyano-pyridin-2-yl)-ureido]-phenoxy}-propyl)-amide;
1-{5-Chloro-2-[3-(3-m-tolyl-ureido)-propoxy]-phenyl}-3-(5-cyano-pyridin-2-yl)-urea;
1-(5-Chloro-2-{3-[3-(4-cyano-phenyl)-ureido]-propoxy}-phenyl)-3-(5-cyano-pyridin-2-yl)-urea;
1-(5-Chloro-2-{3-[3-(4-trifluoromethoxy-phenyl)-ureido]-propoxy}-phenyl)-3-(5-cyano-pyridin-2-yl)-urea;
1-(5-Chloro-2-{3-[3-(2-fluoro-phenyl)-ureido]-propoxy}-phenyl)-3-(5-cyano-pyridin-2-yl)-urea;
1-(5-Chloro-2-{3-[3-(2-methoxy-benzyl)-ureido]-propoxy}-phenyl)-3-(5-cyano-pyridin-2-yl)-urea;
1-(5-Chloro-2-{3-[3-(2-chloro-benzyl)-ureido]-propoxy}-phenyl)-3-(5-cyano-pyridin-2-yl)-urea;
1-(5-Chloro-2-{3-[3-(3,4-difluoro-phenyl)-ureido]-propoxy}-phenyl)-3-(5-cyano-pyridin-2-yl)-urea;
1-(5-Chloro-2-{3-[3-(2,6-dimethyl-phenyl)-ureido]-propoxy}-phenyl)-3-(5-cyano-pyridin-2-yl)-urea;
1-{5-Chloro-2-[3-(3-cyclopentyl-ureido)-propoxy]-phenyl}-3-(5-cyano-pyridin-2-yl)-urea;
1-{5-Chloro-2-[3-(3-cyclohexylmethyl-ureido)-propoxy]-phenyl}-3-(5-cyano-pyridin-2-yl)-urea;
1-(5-Chloro-2-{3-[3-(2-hydroxy-ethyl)-ureido]-propoxy}-phenyl)-3-(5-cyano-pyridin-2-yl)-urea;
1-(5-Chloro-2-{3-[3-(3-dimethylamino-propyl)-ureido]-propoxy}-phenyl)-3-(5-cyano-pyridin-2-yl)-urea;
1-{2-[3-(3-Benzyl-3-methyl-ureido)-propoxy]-5-chloro-phenyl}-3-(5-cyano-pyridin-2-yl)-urea;
1-(2-{3-[3-(2-Amino-ethyl)-ureido]-propoxy}-5-chloro-phenyl)-3-(5-cyano-pyridin-2-yl)-urea;
1-[5-Chloro-2-(3-{3-[2-(1H-imidazol-4-yl)-ethyl]-ureido}-propoxy)-phenyl]-3-(5-cyano-pyridin-2-yl)-urea;
1-[5-Chloro-2-(3-{3-[2-(4-methyl-piperazin-1-yl)-ethyl]-ureido}-propoxy)-phenyl]-3-(5-cyano-pyridin-2-yl)-urea;
1-(5-Chloro-2-{3-[3-(3-pyrrolidin-1-yl-propyl)-ureido]-propoxy}-phenyl)-3-(5-cyano-pyridin-2-yl)-urea;
1-(5-Chloro-2-{3-[3-(4-dimethylamino-benzyl)-ureido]-propoxy-phenyl)-3-(5-cyano-pyridin-2-yl)-urea;
(3-{4-Chloro-2-[3-(5-cyano-pyridin-2-yl)-ureido]-phenoxy}-propyl)-carbamic acid 2-methoxy-ethyl ester;
1-(5-Chloro-2-{3-[3-(2-chloro-ethyl)-ureido]-propoxy}-phenyl)-3-(5-cyano-pyridin-2-yl)-urea;
1-(5-Chloro-2-{3-[3-(2-diethylamino-ethyl)-ureido]-propoxy}-phenyl)-3-(5-cyano-pyridin-2-yl)-urea;
1-(5-Chloro-2-{3-[3-(2-methylamino-ethyl)-ureido]-propoxy}-phenyl)-3-(5-cyano-pyridin-2-yl)-urea;
1-{5-Chloro-2-[3-(3-cyclohexyl-ureido)-propoxy]-phenyl-3-(5-cyano-pyridin-2-yl)-urea;
1-{2-[3-(3-Benzyl-ureido)-propoxy]-5-chloro-phenyl-3-(5-cyano-pyridin-2-yl)-urea;
1-{5-Chloro-2-[3-(3-thiophen-3-yl-ureido)-propoxy]-phenyl-3-(5-cyano-pyridin-2-yl)-urea;
1-(5-Chloro-2-{3-[3-(4-dimethylamino-phenyl)-ureido]-propoxy-phenyl)-3-(5-cyano-pyridin-2-yl)-urea;
(3-{4-Chloro-2-[3-(5-cyano-pyridin-2-yl)-ureido]-phenoxy-propyl)-carbamic acid 2-morpholin-4-yl-ethyl ester;
(S)-2-Amino-N-(3-{4-chloro-2-[3-(5-cyano-pyridin-2-yl)-ureido]-phenoxy}-propyl)-3-(1H-imidazol-4-yl)-propionamide;
(S)-2-Amino-3-(4-amino-phenyl)-N-(3-{4-chloro-2-[3-(5-cyano-pyridin-2-yl)-ureido]-phenoxy-propyl)-propionamide;
(S)-1,2,3,4-Tetrahydro-isoquinoline-3-carboxylic acid (3-{4-chloro-2-[3-(5-cyano-pyridin-2-yl)-ureido]-phenoxy-propyl)-amide;
(S)-Pyrrolidine-2-carboxylic acid (3-{4-chloro-2-[3-(5-cyano-pyridin-2-yl)-ureido]-phenoxy-propyl)-amide;
1-[5-Chloro-2-(4-morpholin-4-yl-4-oxo-butoxy)-phenyl]-3-(5-cyano-pyridin-2-yl)-urea;
2-Amino-N-(3-{4-chloro-2-[3-(5-methyl-pyrazin-2-yl)-ureido]-phenoxy}-propyl)-2-(4-chloro-phenyl)-acetamide; and
pharmaceutically acceptable salts thereof.

17. A pharmaceutical composition comprising the compound of claim 1 and a pharmaceutically acceptable carrier.

18. A method for inhibiting Chk-1, comprising contacting a Chk-1 enzyme with one or more compounds of claim 1 or a pharmaceutically acceptable salt thereof.

19. A method for inhibiting Chk-1 in a subject in need of such inhibition comprising administering to the subject an effective amount of a compound of claim 1.

20. A method for treating cancer in a subject, comprising administering to the subject an effective amount of a compound of claim 1.

21. The method of claim 20, further comprising administering a DNA damaging agent selected from the group consisting of chemotherapy, radiation therapy, and combinations thereof.

22. The method of claim 20, wherein the cancer is multi-drug resistant.

23. The method of claim 20, wherein the cancer is characterized by upregulation of Chk-1 protein or Chk-1 protein activity.

24. A kit comprising (i) a compound of claim 1 or a pharmaceutically acceptable salt thereof; and (ii) a package insert comprising instructions for administering to a subject the compound of claim 1 and a DNA damaging agent.

25. A kit comprising (i) a DNA damaging agent; and (ii) a package insert comprising instructions for administering to a subject the DNA damaging agent and a compound of claim 1 or a pharmaceutically acceptable salt thereof.
